(19) United States
(12) Patent Application Publication
(10) Pub. No.: US 2012/0225889 A1
(43) Pub. Date: Sep. 6, 2012

(54) USE OF MACROCYCLIC LACTONE DERIVATIVES FOR THE TREATMENT OF INFLAMMATORY DISORDERS

(75) Inventors: Asha Kulkarni-Almeida, Goregaon (East) (IN); Ravindra Dattatraya Gupte, Andheri (West) (IN); Nilesh Madhukar Dagia, Goregaon (East) (IN); Prabha Beerchandra Mishra, Goregaon (East) (IN)

(73) Assignee: PIRAMAL LIFE SCIENCES LIMITED, Mumbai (IN)

(21) Appl. No.: 13/510,552

(22) PCT Filed: Nov. 15, 2010
(86) PCT No.: PCT/IB10/55162

§ 371 Date: May 17, 2012

(51) Int. Cl. A61K 31/335; A61P 29/00; A61P 19/02; A61P 19/10; A61P 31/00; A61P 9/10; A61P 9/00; A61P 25/00; A61P 27/02; A61P 31/14; A61P 33/06; A61P 3/00; A61P 17/06; A61P 15/00; A61P 31/18; A61P 37/06; A61P 11/00; A61P 35/00; A61P 3/10; A61P 19/08; A61P 17/00; A61P 25/28; A61P 37/08; A61P 11/06; A61K 31/496; A61K 31/4523; A61K 31/4545; A61P 1/00

(52) U.S. Cl. 514/254.1; 514/450; 514/326; 514/316

(57) ABSTRACT

The present invention provides use of the compounds represented by formula (1): wherein, R₁, R₂, R₃ and R₄ are as defined in the specification, in all their stereo isomeric and tautomeric forms and mixtures thereof in all ratios, and their pharmaceutically acceptable salts, pharmaceutically acceptable solvates, pharmaceutically acceptable polymorphs and prodrugs and pharmaceutical compositions containing them for treatment of inflammatory disorders mediated by one or more cytokines selected from Tumor Necrosis Factor-alpha (TNF-α), interferon-γ (IFN-γ) and interleukins such as IL-1β, IL-2, IL-6, and IL-8. The present invention also relates to a pharmaceutical composition adapted for use in the treatment of inflammatory disorders. The present invention further provides a method of treatment of inflammatory disorders by administering a therapeutically effective amount of the said compound of formula (1) or its pharmaceutical composition, to a mammal in need thereof.

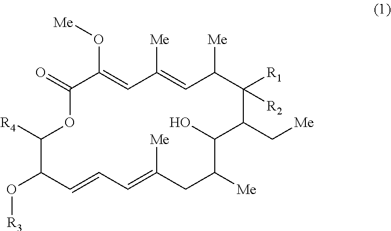

### CROSS-REFERENCE TO RELATED APPLICATION

This application is related to our copending PCT application entitled: “MACROCYCLIC LACTONE DERIVATIVES FOR THE TREATMENT OF CANCER”, filed on the same date as the present application.

### FIELD OF THE INVENTION

The present invention relates to the use of macrocyclic lactone derivatives, and pharmaceutical compositions containing them for the treatment of inflammatory disorders mediated by one or more cytokines selected from Tumor Necrosis Factor-alpha (TNF-α), interferon-γ (IFN-γ) and interleukins such as IL-1β, IL-2, IL-6, and IL-8.

### BACKGROUND OF THE INVENTION

Inflammation is the body's biological response to infection or tissue damage. Under physiological conditions, it is the primary means by which the body fights off invading pathogens and heals the injured tissue. An aberrant inflammatory response can lead to tissue damage and destruction. Chronic uncontrolled inflammation can lead to diseases such as rheumatoid arthritis, osteoarthritis, psoriasis, atherosclerosis, asthma and inflammatory bowel disease (including ulcerative colitis and Crohn's disease).

Rheumatoid arthritis (RA), an autoimmune disorder, is a chronic, systemic, articular inflammatory disease. In addition to joint swelling and pain caused by the inflammatory process, the ultimate hallmark of RA is joint destruction. Indeed, in RA, the normally thin synovial lining of joints is replaced by an inflammatory and highly vascularized, invasive fibrocollagenase tissue (pannus), which is destructive to both cartilage and bone. RA produces its prominent manifestations in the synovial joints. Cartilage destruction in RA is linked to aberrant production of pro-inflammatory cytokines [including tumor necrosis factor-α (TNF-α), interleukin-6 (IL-6) and other interleukins (IL-1β and IL-8)] and growth factor expression in the affected joints.

Psoriasis is an auto-immune/inflammatory disease and although the etiology of psoriasis remains unknown, it is well established that T-cells play a destructive role in psoriasis. Upon getting activated by antigen-presenting cells in the lymph node draining to the skin, T-cells migrate into the skin. In the psoriatic lesions, T-cells release type 1 cytokines [e.g., interleukin-2 (IL-2) and interferon-γ (IFN-α)] and stimulate the neighboring leukocytes. The secreted pro-inflammatory mediators (e.g., TNF-α) drive the hyperproliferation of keratinocytes and, thereby, augment the inflammatory damage in the psoriatic plaque.

Inflammatory bowel disease (IBD) is a group of disorders that cause inflammation of the intestines. The inflammation lasts for a long time and usually relapses. The two major types of IBD are Crohn's disease and ulcerative colitis. Crohn's disease occur when the lining and wall of the intestines becomes inflamed resulting in the development of ulcers. Although Crohn's disease can occur in any part of the digestive system, it often occurs in the lower part of the small intestine where it joins the colon. Ulcerative colitis is a chronic auto-immune/inflammatory disease of unknown etiology afflicting the large intestine. Neither the initiating event nor the sequence of propagating events that lead to and sustain colitis have been fully elucidated. Nevertheless, it is well-established that a dysfunctional immune-response involving components of normal gastrointestinal gram-negative bacteria and increased expression of pro-inflammatory cytokines, chemokines, endothelial cell adhesion molecules (ECAMs) and enhanced leukocyte infiltration into colonic interstitium, play a key role in the pathogenesis of colitis. The course of the disease may be continuous or relapsing, mild or severe. Signs and symptoms of the disease include cramping, lower abdominal pain, rectal bleeding, and frequent, loose discharges consisting mainly of blood, pus, and mucus with scanty fecal particles.

The first line of treatment for inflammatory disorders involves the use of non-steroidal anti-inflammatory drugs (NSAIDs) e.g. ibuprofen, naproxen to alleviate symptoms such as pain. However, despite the widespread use of NSAIDs, many individuals cannot tolerate the doses necessary to treat the disorder over a prolonged period of time as NSAIDs are known to cause gastric erosions. Moreover, NSAIDs merely treat the symptoms of disorder and not the cause. When patients fail to respond to NSAIDs, other drugs such as methotrexate, gold salts, D-penicillamine and corticosteroids are used. These drugs also have significant toxic effects. An increased understanding of the molecular events leading to inflammatory disorders has led to novel approaches for targeting the pathogenesis. Although, the entire sequence of pathobiological events that leads to and sustains various inflammatory disorders is yet to be completely elucidated, it is well established that a milieu of pro-inflammatory cytokines (e.g., TNF-α, IL-6 and IL-1β) play an important role in the pathogenesis of these inflammatory disorders. Indeed, several studies utilizing animal models of inflammation (e.g., RA/colitis/psoriasis) and, more importantly clinically relevant studies of patients with active RA/colitis/psoriasis, have demonstrated that these cytokines are expressed in high-levels both in the inflamed tissue as well as the peripheral blood of patients. These pro-inflammatory mediators induce/up-regulate the expression of adhesion molecules on the microvascular endothelium that leads to recruitment and infiltration of inflammatory leukocytes. Furthermore, in RA settings these cytokines, by virtue of activating osteoclasts, participate in perpetuating inflammation and eventually lead to cartilage degradation and bone erosion. TNF-α, a pleiotropic cytokine, is produced mainly by macrophages. TNF-α demonstrates beneficial as well as pathological activities. It has both growth stimulating effects and growth inhibitory properties, besides being self-regulatory. TNF-α induces the expression of a variety of genes that contribute to various auto-immune/inflammatory disorders.

Although TNF-α plays a critical role in innate and acquired immune responses, an increase in the production of TNF-α can produce pathological changes resulting in chronic inflammation and tissue damage. TNF-α has been shown to play a crucial role in the pathogenesis of many chronic inflammatory disease such as rheumatoid arthritis, juvenile rheumatoid arthritis, psoriatic arthritis, osteoarthritis, refractory rheumatoid arthritis, chronic non-rheumatoid arthritis, osteoporosis/bone resorption, coronary heart disease, vasculitis, inflammatory bowel disease, ulcerative colitis, Crohn's disease, adult respiratory distress syndrome, diabetes, psoriasis, skin delayed type hypersensitivity disorders and Alzheimer's disease. As a specific example, it is well recognized that TNF-α is at the apex of the pro-inflammatory cytokine network in RA. Indeed, it controls the production of other cytokines and orchestrates the inflammatory/immune-response in the synovium. Consistent with this, transgenic mice bearing a de-regulated TNF gene spontaneously develop chronic inflammatory arthritis, and neutralization of TNF-α decreases the incidence and severity of inflammatory arthritis in animal models of RA. These, and several other studies, have demonstrated that TNF-α is an attractive therapeutic target in controlling the aberrant immune/inflammatory response in RA (and also other diseases such as psoriasis and IBD). Most importantly, clinically approved therapies for treating active RA, psoriasis and IBD include TNF-α inhibitors [etanercept (Enbrel), infliximab (Remicade) and adalimumab (Humira)]. In spite of the widespread use of these TNF-α inhibitors, up to 50% of patients treated with TNF blockers fail to improve disease status significantly. Furthermore, while the goal of therapy (in RA patients) is to achieve disease remission and stop joint destruction, existing biologics targeting TNF-α have been shown to stop disease progression in a proportion of, and not all, RA patients.

Interleukin-6 (IL-6) is a pleiotropic cytokine that regulates immunological reactions involved in host defense, inflammation, haematopoiesis, and oncogenesis as reviewed (Blood, 74(1), 1-10, (1989)).

IL-6 has been implicated as a mediator in inflammatory disorders, multiple myelomas, plasmacytomas, Castleman's disease, polyclonal B-cell activation, T cell proliferation, autoimmune disease, AIDS, adult respiratory distress syndrome, cancer, diabetes, ischemia-reperfusion injury, multiple sclerosis, rheumatoid arthritis, and SLE (Blood, 74(1), 1-10, (1989)). Evidence has recently accumulated that overproduction of IL-6 is critically involved in the pathogenesis of RA. Therefore, modulation of this cytokine function may be potentially effective against RA and other chronic and refractory autoimmune/inflammatory diseases. Indeed, anti-interleukin 6 receptor antibody treatment has shown significant efficacy in IL-6 transgenic mice and collagen-induced arthritis (CIA) in DBA/1J mice (Annals of the Rheumatic Diseases, 59 (suppl 1), i21-i27 (2000)).

The therapeutic success of biological inhibitors for TNF-α prompted the development of biological modulators for other targets. Recently, tociluzimab a humanized antibody that binds to both soluble and membrane bound IL-6 receptor has shown exceptional therapeutic efficacy in clinical trials for rheumatoid arthritis. Tocilizumab is approved for treating patients with active RA in Japan and has also gained approval of the FDA's advisory board. Based on this data, interleukin-6 is recommended as a new therapeutic target (Arthritis Research and Therapy, 8(suppl 2), S5, (2006)). Separately, the use of these biologic agents is associated with severe limitations (e.g., parenteral route of administration, high cost of therapy, risk of opportunistic infections, induction of allergic reactions, activation of latent tuberculosis, increased risk of cancer, risk for worsening congestive heart disease). As such, there is an unmet need for small molecule inhibitors of IL-6/TNF-α that would have the same effect as biological agents but without the undesirable side effects.

Intervention of biological activity of IL-6 can be achieved by blocking IL-6 production and/or neutralizating IL-6 (Annals of the Rheumatic Diseases, 59 (suppl 1), i21-i27 (2000)). The etiology of rheumatoid arthritis, inflammatory disorders and other inflammatory conditions is also characterized by uninhibited T-cell proliferation (Arthritis & Rheumatism, 35: 729-735, (1992)). One class of compounds that has received increased attention is compounds inhibiting T-cell proliferation. Limiting T cell activation restricts T cell proliferation as well as the production of the Th1 cytokines, which are implicated in activation of the monocyte macrophage system in the rheumatoid or synovial milieu (Arthritis Research, 4 (suppl 3):S197-S211, (2002)).

T cell activation is marked by the expression of specific proteins that aid in their effect or functions. Among the first proteins to be expressed are interleukin-2 (IL-2) and IL-2 receptor alpha subunit. IL-2 is a potent T cell mitogen, which is required for T cell proliferation. IL-2 signaling is required for T cells to initiate the immune response. IL-2 is a potent T cell growth cytokine, which, in T cell activation, acts in an autocrine fashion to promote the growth, proliferation and differentiation of the T cell which has been recently stimulated by antigen. Indeed, T cells that receive inappropriate signaling become anergic i.e. they become inactive. This is accomplished by making the T cell unable to synthesize IL-2. This renders them potentially inert to any antigenic stimulation they might receive in the future. Clinically compounds such as cyclosporin, FK506 and rapamycin are known to induce anergy to continuous antigenic stimulus by blocking this IL-2 signaling via distinct pathways.

A strong association between major histocompatibility complex (MHC) alleles expressed on T cells and synovial macrophages dictates the progress of rheumatoid arthritis. These T cell effector responses are driven by antigen expression on synovial cell macrophages and dendritic cells. Contact mediated T cell monocyte interaction drives the stimulation of the proinflammatory cytokine cascade in the synovial cell joints (Arthritis Research, 4 (suppl 3): S169-S176, (2002)) Based on the type of stimulus T cells further differentiate into T helper type 1 (Th1) and T helper type 2 (Th2) cells. These cells are characterized on the basis of their cytokine expression profile. Th1 cells secrete IFN-γ and TNF-α whereas Th2 cells secrete IL-4, IL-5 and IL-10 (Nature Immunology, 7(3): 247-255, (2006)).

The Th1 cells enhance macrophage activation and drive further activation of the inflammatory cellular cascade. This signaling is enhanced by interactions between co-stimulatory molecules (B7.1, B7.2, B7.3) expressed on the cell surface of both cell types. These interactions involve secretion of IFN-γ and TNF-α (Nature Immunology, 2(3): 269-274, (2001)). All proliferating T cells constitutively express IL-2, thus inhibiting these cytokines offers a putative negative signal for the progression of any inflammatory condition. Thus compounds blocking T cell proliferation have a better potential to restrict inflammatory disorders. Such compounds may also exhibit immunosuppressive properties. FK506 (tacrolimus) is an immunosuppressive agent that specifically suppresses T cell activation. FK506 exerts its immunosuppressive effects after binding to intracellular proteins, termed FK506 binding proteins (FKBPs) (J. Antibiotics, 40, 1256-1265, (1987), J. Immunology, 139, 1797-1803, (1987)). FK506 was also efficacious in the treatment of CIA. Possibly, FK506 suppresses paw swelling and prevents bone and cartilage destruction in CIA by inhibiting T cell activation and subsequent production of inflammatory cytokines, such as TNF-α.

The compounds described in the present invention inhibit T-cell proliferation and block production of the cytokines. These effects may be contributing towards their therapeutic efficacy.

Transcriptional coactivators have crucial roles in eukaryotic transcription. One of the factors that can activate transcription factors in macrophages is bacterial lipopolysaccharide (LPS). Bacterial endotoxin such as LPS is known to be one of the inducers of macrophagic activation. Activation of macrophages is involved in augmentation of several inflammatory conditions; e.g., rheumatoid arthritis, inflammatory bowel disease, sepsis and other diseases. LPS activation of macrophages triggers Toll-like receptor 4 (TLR4). TLR4 is a protein that in humans is encoded by the TLR4 gene. TLR4 signalling and activation of TLRs is associated with induction of pro-inflammatory gene expression.

LPS also activates the transcription factor nuclear factor-kB (NF-kB) through the IkB kinase complex (IKK). NF-kB is a transcription factor that controls inflammation and host responses (The Journal of Biological Chemistry Vol. 281, No. 41, 31142-31151, (2006)). Besides, NF-kB, a large family of nuclear transcription factors, the interferon regulatory factors (IRFs), have been implicated in TLR signaling leading to pro-inflammatory gene expression (Journal of Leukocyte Biology, Vol. 83, 1249-1257, (2008)). IRF-1 and IRF-2 counter-regulate the transcriptional activity of many genes. Thus IRF-1 and IRF-2 induced signaling involves cAMP response element binding protein (CREB) pathway. One of the coactivators, CREB binding protein (CBP), regulates gene expression with a number of transcription factors via two mechanisms. One is the recruitment of general transcriptional machinery to the promoters. The presence of CBP complexes in rheumatoid synoviocytes has been reported (Mod Rheumatol., 14, 6-11, (2004)). These associations may regulate proliferation and apoptosis in RA patients. CREB activation has also been previously reported to be induced by mitochondrial dysfunction and is implicated in signaling cell proliferation (EMBO Journal, 21, Nos. 1 and 2, 53-63, (2002)). The promoter region of human IL-6 has four major binding sites (FEBS Letters, 541, 3, 33-39, (2003)). Two members of CEBP family i.e. C/EBPβ and C/EBPδ are collectively responsible for IL-6 transcription (Cellscience Reviews, Vol. 2, No. 2, ISSN 1742 (2005)) and are the transcriptional target of cAMP mediated phosphorylation of CREB (Am. J. Physiol. Regul. Integr. Comp. Physiol. 283: R1140-R1148, (2002)), Int. J. Biochm. Cell Bid. Vol. 29 No. (12). 1401 1418. (1997)). The promoter region of human IL-6 is having four major binding sites. MRE region (for TNF-α, IL-1 and Forskolin binding), NF-kB binding region, API binding region, and C/EBPb binding region.

Matrix metallo proteins (MMPs) play an important role in RA and various MMPs like MMP1, MMP3, MMP13 and TIMP2 are overexpressed in RA (Ann Rheum Dis, 69, 898-902, (2010), Biochimica et Biophysica Acta, 1502, 307-318, (2000)). Myeloid differentiation primary response gene (88) (MyD88), a TLR4 dependent protein is known to be constitutively expressed by rheumatoid synovial cells (Rheumatology, 45, 527-532, (2006)). MMP13, Guanylate binding protein 1 (GBP-1) and MyD88 contribute towards the inflammatory destructive processes in RA and hence are critical signaling molecules for determining the therapeutic index of a treatment regimen.

### SUMMARY OF THE INVENTION

The present invention relates to the use of macrocyclic lactone derivatives for the treatment of an inflammatory disorder mediated by one or more cytokines selected from Tumor Necrosis Factor-alpha (TNF-α), interferon-γ (IFN-γ) and interleukins such as IL-1β, IL-2, IL-6, and IL-8.

According to a further aspect, there is provided the use of compound of formula (1) (as provided herein below), for the treatment of an inflammatory disorder mediated by one or more cytokines selected from Tumor Necrosis Factor-alpha (TNF-α), interferon-γ (IFN-γ) and interleukins such as IL-1, IL-2, IL-6, and IL-8.

According to another aspect of the present invention, there are provided pharmaceutical compositions including one or more compounds of formula (1) as active ingredient, for the treatment of an inflammatory disorder mediated by one or more cytokines selected from Tumor Necrosis Factor-alpha (TNF-α), interferon-γ (IFN-γ) and interleukins such as IL-1β, IL-2, IL-6, and IL-8.

According to another aspect of the present invention, there is provided a method for the treatment of an inflammatory disorder mediated by one or more cytokines selected from Tumor Necrosis Factor-alpha (TNF-α), interferon-γ (IFN-γ) and interleukins such as IL-1β, IL-2, IL-6, and IL-8 the method including administering to a mammal in need thereof, a therapeutically effective amount of one or more compounds of general formula (1).

According to another aspect of the present invention, there are provided methods for the manufacture of medicaments including one or more compounds of formula (1) which are useful for the treatment of an inflammatory disorder mediated by one or more cytokines selected from Tumor Necrosis Factor-alpha (TNF-α), interferon-γ (IFN-γ) and interleukins such as IL-1β, IL-2, IL-6, and IL-8.

According to still another aspect of the invention, there are provided methods for the treatment of an inflammatory disorder by down-regulating one or more genes selected from BCL2, CEBPα, CEBPβ, CEBPδ, IL-1β, IL-6, cMyc, GBP-1, MMP13 and MyD88.

According to another aspect of the invention, there is provided a method for monitoring drug response in a patient with an inflammatory disorder treated with a compound of formula (1), comprising determining the expression of one or more genes selected from CEBPα, CEBPβ, CEBPδ, IL-1β, IL-6, GBP-1, MMP 13, MyD88, BCL2 and cMyc in a sample from the patient.

### DETAILED DESCRIPTION OF THE INVENTION

The present invention provides compounds represented by the following formula (1):

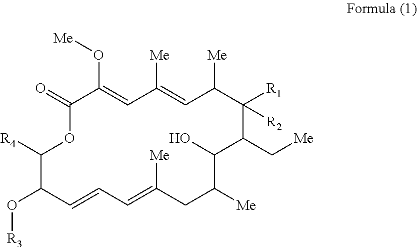

and all of their stereoisomeric and tautomeric forms and mixtures thereof, in all ratios, and their pharmaceutically acceptable salts, pharmaceutically acceptable solvates, pharmaceutically acceptable polymorphs and prodrugs,
wherein,
R₁ is selected from halogen, hydroxy, alkoxy, —O(CO)R₁₃, —SR₁₄, and —NR₁₄R₁₅;
R₂ is hydrogen; or
optionally R₁ is absent and R₂ is ═O;
R₃ is alkyl;
R₄ is selected from the following formulae:

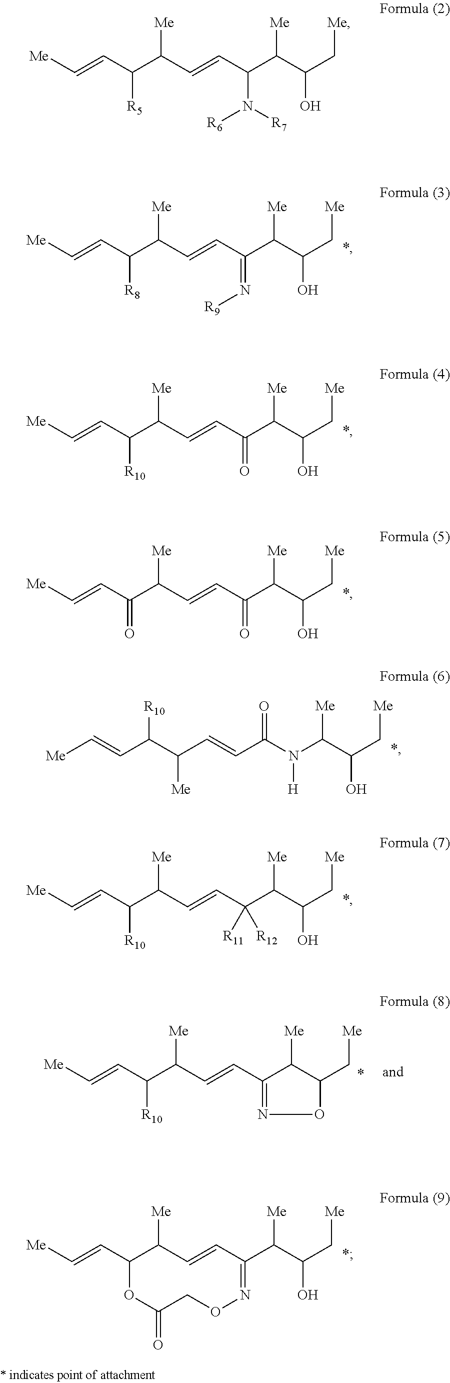

R₅ is selected from hydroxy, and alkoxy;
R₆ is selected from hydrogen, hydroxy, alkyl, and alkoxy;
R₇ is selected from hydrogen, alkyl, and —(CO)R₁₆;
R₈ is selected from hydroxy, and alkoxy;
R₉ is selected from hydroxy, alkyl, alkoxy, aryl, aralkyl, aryloxy, benzyloxy, heterocyclyl, —O-heterocyclyl, —OCH₂COOR₁₇, and —OCH₂COR₁₈;
R₁₀ is selected from halogen, hydroxy, alkoxy, —SR₁₄, —NR₁₄R₁₅, and —O(CO)R₁₉;
R₁₁ is selected from hydrogen, and halogen;
R₁₂ is selected from hydrogen, halogen, and hydroxy;
R₁₃ is selected from alkyl, and aryl;
R₁₄ is selected from hydrogen, alkyl, aralkyl, aryl, and heterocyclyl;
R₁₅ is selected from hydrogen, and alkyl;
R₁₆ is selected from alkyl, and aryl;
R₁₇ is selected from hydrogen, and alkyl;
R₁₈ is selected from alkyl, —NHCH₂R₂₀, aryl, and heterocyclyl;
R₁₉ is selected from alkyl, aralkyl, aryl, and heterocyclyl; and
R₂₀ is selected from hydrogen, alkyl, aryl, and heterocyclyl;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, alkoxy, aryl, and heterocyclyl;
heterocyclyl is unsubstituted or substituted by one or two of the same or different groups selected from: halogen, hydroxy, amino, alkyl, hydroxyalkyl, alkoxy, aryl, and heterocyclyl; for the treatment of an inflammatory disorder mediated by one or more cytokines selected from Tumor Necrosis Factor-alpha (TNF-α), interferon-γ (IFN-γ) and interleukins such as IL-113, IL-2, IL-6.

### DEFINITIONS

Listed below are definitions, which apply to the terms as they are used throughout the specification and the appended claims (unless they are otherwise limited in specific instances), either individually or as part of a larger group.

As used herein, the term “alkyl” whether used alone or as part of a substituent group, refers to saturated aliphatic groups, including straight or branched-chain containing from 1 to 6 carbon atoms. Suitable alkyl groups contain for example, from 1 to 4 carbon atoms, such as methyl, ethyl, propyl, isopropyl, n-butyl, isobutyl, and t-butyl. An alkyl group is optionally substituted by one or more identical or different substituents. Any kind of substituent present in substituted alkyl groups can be present in any desired position provided that the substitution does not lead to an unstable molecule. A substituted alkyl refers to an alkyl group in which one or more, for example, 1, 2, 3, 4 or 5 hydrogen atoms are replaced with substituents, for example, halogen, hydroxy, amino, alkoxy, hydroxyalkyl, aryloxy, acyloxy, aryl, heteroaryl, or heterocyclyl group.

As used herein, the term “alkoxy” refers to an alkyl group having an oxygen attached thereto, wherein alkyl is as defined above. Representative alkoxy groups include methoxy, ethoxy, propoxy, and tert-butoxy group. The terms include, therefore, alkoxy groups, which are substituted by one or more identical or different groups selected from: halogen, hydroxy, alkyl, and hydroxyalkyl.

As used herein, the term “aryl” refers to a monocyclic or bicyclic hydrocarbon group having up to 10 ring carbon atoms, in which at least one carbocyclic ring is present that has a conjugated π electron system. Examples of aryl group include phenyl and naphthyl. A substituted aryl refers to an aryl group, which is substituted by one or more substituents, for example, up to five identical or different substituents selected from the group consisting of halogen, hydroxy, amino, alkyl, hydroxyalkyl, alkoxy, aryloxy, aryl, and a heterocyclyl group. Aryl groups can be substituted in any desired position. For example, in monosubstituted phenyl groups, the substituent can be located in the 2-position, the 3-position, the 4-position or the 5-position. If the phenyl group carries two substituents, they can be located in 2,3-position, 2,4-position, 2,5-position, 2,6-position, 3,4-position or 3,5-position.

As used herein, the term “aryloxy” refers to the aryl-O— wherein the term aryl is as defined above. Exemplary aryloxy groups include, but are not limited to, phenoxy and naphthoxy. The term “heteroatom” refers to nitrogen, oxygen and sulfur. It should be noted that any heteroatom with unsatisfied valences is assumed to have a hydrogen atom to satisfy the valences. The ring heteroatoms can be present in any desired number and in any position with respect to each other provided that the resulting heterocyclic system is stable.

The terms “heterocyclyl”, and “heterocyclic” refer to a saturated, partially unsaturated or aromatic monocyclic or bicyclic ring system containing 3, 4, 5, 6, 7, 8, 9, or 10, ring atoms of which 1, 2, 3 or 4 are identical or different heteroatoms selected from: nitrogen, oxygen and sulfur. The heterocyclyl group may, for example, have 1 or 2 oxygen atoms and/or 1 or 2 sulfur atoms and/or 1 to 4 nitrogen atoms in the ring. Heterocyclyl includes saturated heterocyclic ring systems, which do not contain any double bonds within the rings, as well as unsaturated heterocyclic ring systems, which contain one or more, up to 5 double bonds within the rings provided that the resulting system is stable. Unsaturated rings may be non-aromatic or aromatic. Aromatic heterocyclyl groups may also be referred to by the customary term “heteroaryl” for which all the definitions and explanations above and below relating to heterocyclyl apply. Monocyclic heterocyclyl groups include 3-membered, 4-membered, 5-membered, 6-membered and 7-membered rings. Suitable examples of such heterocyclyl groups are pyrrolyl, imidazolyl, pyrrolidinyl, pyridinyl, pyrazinyl, pyridazinyl, pyrimidinyl, pyrazolyl, triazolyl, tetrazolyl, piperidinyl, piperazinyl, and morpholinyl. Bicyclic heterocyclyl groups include two fused rings, one of which is 5-, 6- or 7-membered heterocyclic ring and the other of which is a 5-, 6- or 7-membered carbocyclic or heterocyclic ring. Exemplary bicyclic heterocyclic groups include benzoxazolyl, quinolyl, isoquinolyl, indolyl, isoindolyl, and benzofurazanyl.

A substituted heterocyclyl refers to a heterocyclyl group which is substituted with one or more (up to 5), identical or different substituents. Examples of substituents for the ring carbon and ring nitrogen atoms are: halogen, hydroxy, amino, alkyl, hydroxyalkyl, alkoxy, aryloxy, aryl, and heterocyclyl. The substituents can be present at one or more positions provided that a stable molecule results.

As used herein the term “aralkyl” refers to an alkyl group substituted with an aryl or heteroaryl group, wherein the terms alkyl, aryl and heteroaryl are as defined above. Exemplary aralkyl groups include —(CH₂)ₚ-phenyl, —(CH₂)ₚ-pyridyl, wherein p is an integer from 1 to 3. The aralkyl group may be further substituted with hydroxy, halogen, amino, alkyl, aryl or heteroaryl.

As used herein, the term —O-heterocyclyl refers to the heterocyclic ring attached directly to an oxygen atom wherein the term heterocyclyl is as defined above.

The term “halogen” refers to fluorine, chlorine, bromine or iodine.

The term “amino” refers to unsubstituted, mono-substituted and di-substituted amino groups.

As used herein, the terms mono- or di-substituted amino refer respectively to an amino group substituted by one or two groups which may be the same or different. The substituents on the amino group are independently selected from: alkyl, hydroxyalkyl, aralkyl, aryl, and heterocyclyl. It will be understood by those skilled in the art that the moieties on the amino group can themselves be substituted, if appropriate.

The expression “prodrug” refers to compounds that are drug precursors, which following administration, release the drug in vivo via a chemical or physiological process e.g., a prodrug on being brought to the physiological pH or through an enzyme action is converted to the desired drug form.

It will be understood that “substitution” or “substituted with” includes the implicit proviso that such substitution is in accordance with permitted valence of the substituted atom and the substituent, as well as results in a stable compound, which does not readily undergo transformation such as by rearrangement, cyclization, elimination, etc.

The term “subject” as used herein refers to an animal, preferably a mammal, and most preferably a human.

The term “mammal” used herein refers to warm-blooded vertebrate animals of the class Mammalia, including humans, characterized by a covering of hair on the skin and, in the female, milk-producing mammary glands for nourishing the young. The term mammal includes animals such as cat, dog, rabbit, bear, fox, wolf, monkey, deer, mouse, pig as well as human.

The term “test sample” refers to a biological material suspected of containing the analyte. The test sample may be derived from any biological source, such as a physiological fluid, including, blood, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen, bile, cerebrospinal fluid, feces, gastric or intestinal secretions and so forth.

Preferred types of samples are blood and synovial fluid.

The term “treating”, “treat” or “treatment” as used herein refers to alleviate, slow the progression, attenuation or cure of existing disease (for example, rheumatoid arthritis).

By “pharmaceutically acceptable” it is meant that the carrier, diluent, excipients, and/or salt must be compatible with the other ingredients of the formulation, and not deleterious to the recipient thereof.

The term “pharmaceutically acceptable carrier” as used herein means a non-toxic, inert, solid, semi-solid, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose and cellulose acetate; malt; gelatin; talc; as well as other non-toxic compatible lubricants such as sodium lauryl sulfate and magnesium stearate, as well as coloring agents, releasing agents, coating agents, sweetening, flavoring and perfuming agents; preservatives and antioxidants can also be present in the composition, according to the judgment of the formulator.

The term, “therapeutically effective amount” as used herein means an amount of compound or composition (e.g. compound of formula (1)) sufficient to significantly induce a positive modification in the condition to be regulated or treated, but low enough to avoid undue or severe side effects, within the scope of sound medical judgment. The therapeutically effective amount of the compound or composition will vary with the particular condition being treated, the age and physical condition of the end user, the severity of the condition being treated/prevented, the duration of the treatment, the nature of concurrent therapy, the specific compound or composition employed, the particular pharmaceutically acceptable carrier utilized, and like factors. As used herein, all percentages are by weight unless otherwise specified.

The term “abnormal” as used herein and in the appended claims in the context of one or more proinflammatory cytokines selected from Tumor Necrosis Factor-alpha (TNF-α), interferon-γ (IFN-γ) and interleukins such as IL-1β, IL-2, IL-6 and IL-8 refers to elevated or increased levels of the proinflammatory cytokines.

### EMBODIMENTS OF THE INVENTION

The present invention provides compounds represented by the following formula (1),

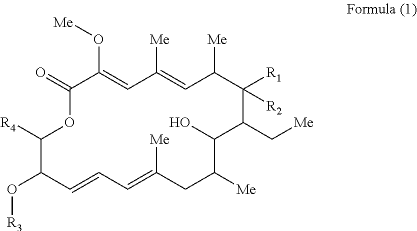

and all of their stereoisomeric and tautomeric forms and mixtures thereof, in all ratios, and their pharmaceutically acceptable salts, pharmaceutically acceptable solvates, pharmaceutically acceptable polymorphs and prodrugs,
wherein,
R₁ is selected from halogen, hydroxy, alkoxy, —O(CO)R₁₃, —SR₁₄, and —NR₁₄R₁₅;
R₂ is hydrogen;
R₃ is alkyl;
R₄ is selected from the following formulae:

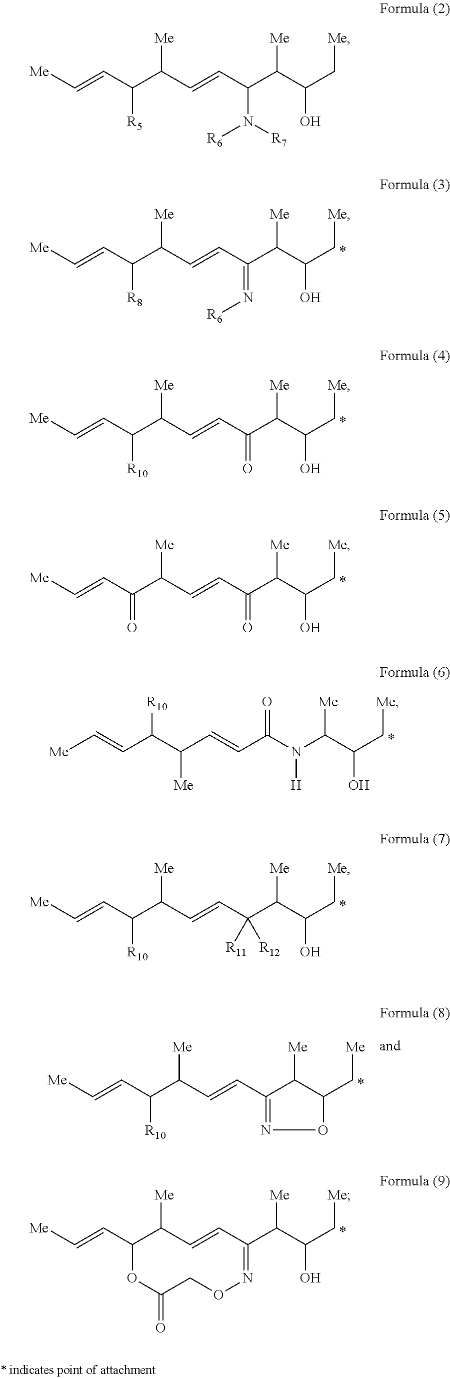

R₅ is selected from hydroxy, and alkoxy;
R₆ is selected from hydrogen, hydroxy, alkyl, and alkoxy;
R₇ is selected from hydrogen, alkyl, and —(CO)R₁₆;
R₈ is selected from hydroxy, and alkoxy;
R₉ is selected from hydroxy, alkyl, alkoxy, aryl, aralkyl, aryloxy, benzyloxy, heterocyclyl, —O-heterocyclyl, —OCH₂COOR₁₇, and —OCH₂COR₁₈;
R₁₀ is selected from halogen, hydroxy, alkoxy, —SR₁₄, —NR₁₄R₁₅, and —O(CO)R₁₉;
R₁₁ is selected from hydrogen, and halogen;
R₁₂ is selected from hydrogen, halogen, and hydroxy;
R₁₃ is selected from alkyl, and aryl;
R₁₄ is selected from hydrogen, alkyl, aralkyl, aryl, and heterocyclyl;
R₁₅ is selected from hydrogen, and alkyl;
R₁₆ is selected from alkyl, and aryl;
R₁₇ is selected from hydrogen, and alkyl;
R₁₈ is selected from alkyl, —NHCH₂R₂₀, aryl, and heterocyclyl;
R₁₉ is selected from alkyl, aralkyl, aryl, and heterocyclyl; and
R₂₀ is selected from hydrogen, alkyl, aryl, and heterocyclyl;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, alkoxy, aryl, and heterocyclyl; heterocyclyl is unsubstituted or substituted by one or two of the same or different groups selected from: halogen, hydroxy, amino, alkyl, hydroxyalkyl, alkoxy, aryl, and heterocyclyl; for the treatment of an inflammatory disorder mediated by one or more cytokines selected from Tumor Necrosis Factor-alpha (TNF-α), interferon-γ (IFN-γ) and interleukins such as IL-1(i, IL-6, and IL-8.

In one embodiment, the present invention provides compounds represented by formula (1), wherein,

R₁ is selected from halogen, hydroxy, and alkoxy;
R₂ is hydrogen;
R₃ is alkyl;
R₄ is formula (3):

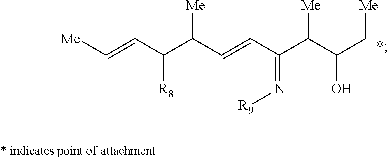

R₈ is hydroxy;
R₉ is selected from hydroxy, alkyl, alkoxy, aryl, aralkyl, aryloxy, benzyloxy, —OCH₂COOR₁₇, and —OCH₂COR₁₈;
R₁₇ is selected from hydrogen, and alkyl;
R₁₈ is selected from alkyl, —NHCH₂R₂₀, aryl, and heterocyclyl; and
R₂₀ is selected from hydrogen, alkyl, aryl, and heterocyclyl;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, alkoxy, aryl, and heterocyclyl;
heterocyclyl is unsubstituted or substituted by one or two of the same or different groups selected from: halogen, hydroxy, amino, alkyl, hydroxyalkyl, alkoxy, aryl, and heterocyclyl.

In a further embodiment, the present invention provides compounds represented by formula (1), wherein,

R₁ is hydroxy;
R₂ is hydrogen;
R₃ is alkyl;
R₄ is formula (3):

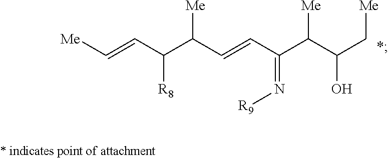

R₈ is hydroxy; and
R₉ is selected from hydroxy, alkyl, alkoxy, and benzyloxy;
where alkyl is unsubstituted or substituted by one or two of the same or different groups selected from: hydroxy, halogen, amino, hydroxyalkyl, and alkoxy.

In a further embodiment, the present invention provides compounds represented by formula (1), wherein,

R₁ is hydroxy;
R₂ is hydrogen;
R₃ is methyl;
R₄ is formula (3):

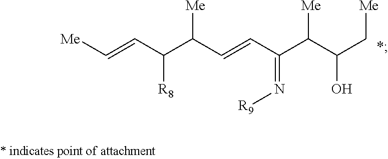

R₈ is hydroxy; and
R₉ is selected from hydroxy, methoxy, and benzyloxy.

In a further embodiment, the present invention provides compound represented by formula (1), wherein,

R₁ is hydroxy;
R₂ is hydrogen;
R₃ is methyl;
R₄ is formula (3):

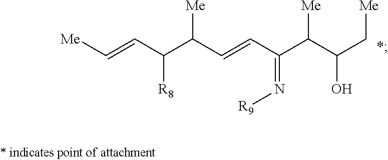

R₈ is hydroxy; and
R₉ is hydroxy.

In one embodiment, the present invention provides compounds represented by formula (1), wherein,

R₁ is selected from halogen, hydroxy, and alkoxy;
R₂ is hydrogen;
R₃ is alkyl;
R₄ is formula (3):

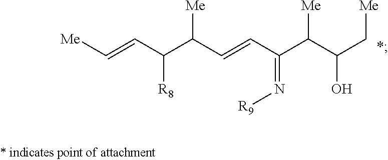

R₈ is selected from hydroxy, and alkoxy;
R₉ is selected from —OCH₂COOR₁₇, and —OCH₂COR₁₈;
R₁₇ is selected from hydrogen, and alkyl;
R₁₈ is selected from alkyl, heterocyclyl and —NHCH₂R₂₀; and
R₂₀ is selected from hydrogen, alkyl, aryl, and heterocyclyl;
where alkyl is unsubstituted or substituted by one or two of the same or different groups selected from: hydroxy, halogen, amino, hydroxyalkyl, and alkoxy;
alkoxy is unsubstituted or substituted by one or two of the same or different groups selected from: hydroxy, alkyl, and hydroxyalkyl;
aryl is unsubstituted or substituted by one or two of the same or different groups selected from: halogen, hydroxy, hydroxyalkyl, alkoxy, aryl, and heterocyclyl;
heterocyclyl is unsubstituted or substituted by one or two of the same or different groups selected from: hydroxy, hydroxyalkyl, alkyl, alkoxy, aryl, and heterocyclyl.

In a further embodiment, the present invention provides compounds represented by formula (1), wherein,

R₁ is hydroxy;
R₂ is hydrogen;
R₃ is methyl;
R₄ is formula (3):

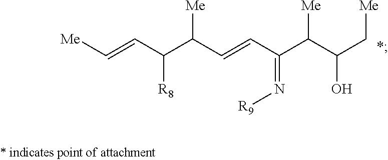

R₈ is hydroxy;

### R₉ is —OCH₂COOR₁₇; and

R₁₇ is selected from hydrogen, and alkyl.

In one embodiment, the present invention provides compounds represented by formula (1), wherein,

R₁ is hydroxy;
R₂ is hydrogen;
R₃ is methyl;
R₄ is formula (3):

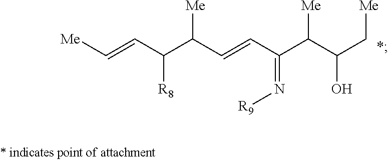

R₈ is hydroxy;

### R₉ is —OCH₂COR₁₈; and

R₁₈ is selected from 4-methylpiperazin-1-yl, piperidin-1-yl, and 1,4′-bipiperidin-1′-yl.

In one embodiment, the present invention provides compounds represented by formula (1), wherein,

R₁ is hydroxy;
R₂ is hydrogen;
R₃ is methyl;
R₄ is formula (3):

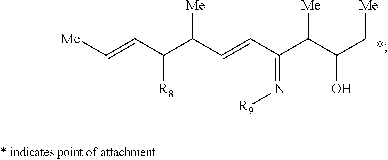

R₈ is hydroxy;

### R₉ is —OCH₂COR₁₈;

### R₁₈ is —NHCH₂R₂₀; and

R₂₀ is selected from alkyl, and aryl;
where alkyl is unsubstituted or substituted by one or two of the same or different groups selected from: hydroxy, halogen, amino, hydroxyalkyl, and alkoxy;
aryl is unsubstituted or substituted by one or two of the same or different groups selected from: halogen, hydroxy, and alkoxy.

In one embodiment, the present invention provides compounds represented by formula (1), wherein,

R₁ is hydroxy;
R₂ is hydrogen;
R₃ is methyl;
R₄ is formula (3):

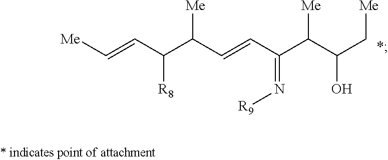

R₈ is hydroxy;

### R₉ is —OCH₂COR₁₈;

### R₁₈ is —NHCH₂R₂₀; and

R₂₀ is selected from —CH₂OH, and 4-fluorophenyl.

In another embodiment, the present invention provides compounds represented by formula (1), wherein

R₁ is selected from halogen, hydroxy, and alkoxy;
R₂ is hydrogen;
R₃ is alkyl;
R₄ is formula (6):

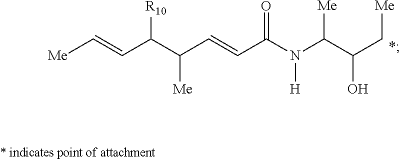

R₁₀ is selected from halogen, hydroxy, and alkoxy;
where alkyl is unsubstituted or substituted by one or two of the same or different groups selected from: hydroxy, halogen, amino, hydroxyalkyl, and alkoxy;
alkoxy is unsubstituted or substituted by one or two of the same or different groups selected from: halogen, hydroxy, alkyl, and hydroxyalkyl.

In one embodiment, the present invention provides compounds represented by formula (1), wherein,

R₁ is hydroxy;
R₂ is hydrogen;
R₃ is methyl;
R₄ is formula (6):

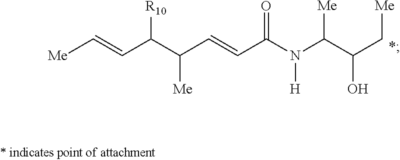

R₁₀ is hydroxy, and alkoxy.

In another embodiment, the present invention provides compounds represented by formula (1), wherein,

R₁ is selected from halogen, hydroxy, and alkoxy;
R₂ is hydrogen;
R₃ is alkyl;
R₄ is formula (7):

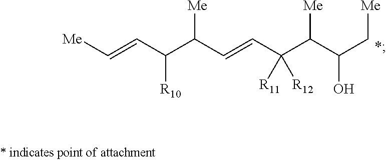

R₁₀ is selected from halogen, hydroxy, and alkoxy;
R₁₁ is selected from hydrogen, and halogen; and
R₁₂ is selected from hydrogen, halogen, and hydroxy.

In another embodiment, the present invention provides compounds represented by formula (1), wherein,

R₁ is hydroxy;
R₂ is hydrogen;
R₃ is methyl;
R₄ is formula (7):

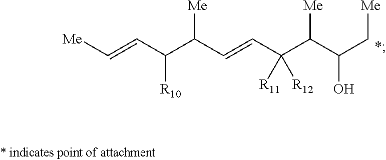

R₁₀ is hydroxy, and alkoxy;
R₁₁ is hydrogen; and
R₁₂ is hydroxy.

In another embodiment, the present invention provides compounds represented by formula (1), wherein,

R₁ is hydroxy;
R₂ is hydrogen;
R₃ is methyl;
R₄ is formula (7):

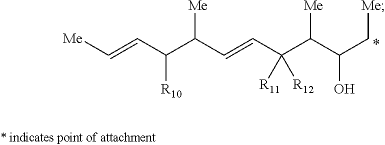

R₁₀ is hydroxy, and alkoxy;
R₁₁ is halogen; and
R₁₂ is halogen.

In one embodiment, the present invention provides compounds represented by formula (1), wherein,

R₁ is selected from halogen, hydroxy, and alkoxy;
R₂ is hydrogen;
R₃ is alkyl;
R₄ is formula (2):

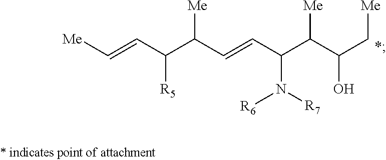

R₅ is selected from hydroxy, and alkoxy;
R₆ is selected from hydrogen, alkyl, hydroxy, and alkoxy;
R₇ is selected from hydrogen, alkyl, and —(CO)R₁₆; and
R₁₆ is selected from alkyl, and aryl;
where alkyl is unsubstituted or substituted by one or two of the same or different groups selected from: hydroxy, halogen, amino, hydroxyalkyl, and alkoxy;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, alkoxy, aryl, and heterocyclyl.

In one embodiment, the present invention provides compounds represented by formula (1), wherein,

R₁ is selected from halogen, hydroxy, and alkoxy;
R₂ is hydrogen;
R₃ is alkyl;
R₄ is formula (2):

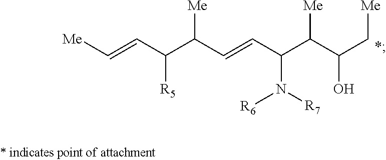

R₅ is selected from hydroxy, and alkoxy;
R₆ is selected from hydrogen, and hydroxy;
R₇ is selected from hydrogen, alkyl, and —(CO)R₁₆; and
R₁₆ is alkyl;
where alkyl is unsubstituted or substituted by one or two of the same or different groups selected from: hydroxy, halogen, amino, hydroxyalkyl, and alkoxy;
alkoxy is unsubstituted or substituted by one or two of the same or different groups selected from: halogen, hydroxy, alkyl, and hydroxyalkyl.

In one embodiment, the present invention provides compounds represented by formula (1), wherein,

R₁ is hydroxy;
R₂ is hydrogen;
R₃ is alkyl;
R₄ is formula (2):

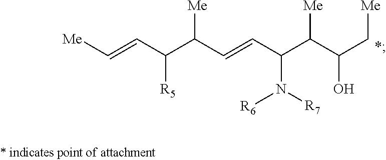

R₅ is selected from hydroxy, and alkoxy;
R₆ is hydrogen;
R₇ is selected from hydrogen and —(CO)R₁₆; and
R₁₆ is alkyl.

In one embodiment, the present invention provides compounds represented by formula (1), wherein,

R₁ is selected from halogen, hydroxy, alkoxy, —O(CO)R₁₃, —SR₁₄, and —NR₁₄R₁₅;
R₂ is hydrogen;
R₃ is alkyl;
R₄ is formula (4):

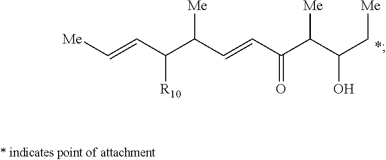

R₁₀ is selected from halogen, hydroxy, alkoxy, —SR₁₄, —NR₁₄R₁₅, and —O(CO)R₁₉;
R₁₃ is selected from alkyl, and aryl;
R₁₄ is selected from hydrogen, alkyl, aralkyl, aryl, and heterocyclyl;
R₁₅ is selected from hydrogen, and alkyl; and
R₁₉ is selected from alkyl, aryl, and heterocyclyl;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, alkoxy, aryl, and heterocyclyl;
heterocyclyl is unsubstituted or substituted by one or two of the same or different groups selected from: halogen, hydroxy, amino, alkyl, hydroxyalkyl, alkoxy, aryl, and heterocyclyl.

In one embodiment, the present invention provides compounds represented by formula (1), wherein,

### R₁ is —SR₁₄;

R₂ is hydrogen;
R₃ is methyl;
R₄ is formula (4):

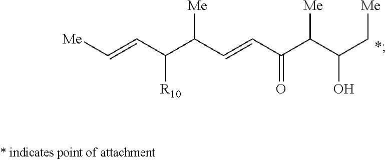

### R₁₀ is —SR₁₄; and

R₁₄ is selected from hydrogen, and alkyl;
where alkyl is unsubstituted or substituted by one or two of the same or different groups selected from: hydroxy, halogen, amino, hydroxyalkyl, and alkoxy.

In one embodiment, the present invention provides compounds represented by formula (1), wherein,

### R₁ is —NR₁₄R₁₅;

R₂ is hydrogen;
R₃ is alkyl;
R₄ is formula (4):

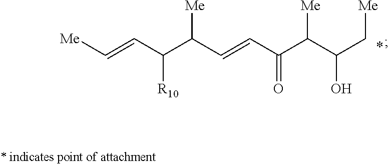

R₁₀ is selected from halogen, hydroxy, alkoxy, —SR₁₄, —NR₁₄R₁₅, and —O(CO)R₁₉;
R₁₄ is selected from hydrogen, alkyl, aralkyl, aryl, and heterocyclyl;
R₁₅ is selected from hydrogen, and alkyl; and
R₁₉ is selected from alkyl, aryl, and heterocyclyl;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, and alkoxy;
heterocyclyl is unsubstituted or substituted by one or two of the same or different groups selected from: halogen, hydroxy, amino, alkyl, hydroxyalkyl, and alkoxy.

In one embodiment, the present invention provides compounds represented by formula (1), wherein,

### R₁ is —NR₁₄R₁₅;

R₂ is hydrogen;
R₃ is alkyl;
R₄ is formula (4):

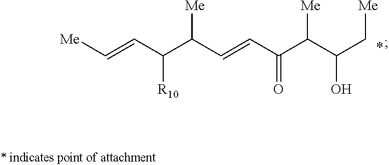

### R₁₀ is —NR₁₄R₁₅;

R₁₄ is selected from hydrogen, and alkyl; and
R₁₅ is selected from hydrogen, and alkyl;
where alkyl is unsubstituted or substituted by one or two of the same or different groups selected from: hydroxy, halogen, amino, hydroxyalkyl, and alkoxy;

In one embodiment, the present invention relates to the use of a compound of formula (1), wherein,

R₁ is hydroxy;
R₂ is hydrogen;
R₃ is alkyl;
R₄ is formula (4):

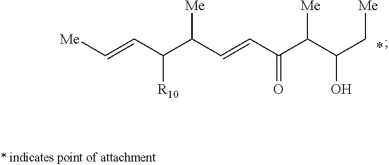

R₁₀ is hydroxy.

In one embodiment, the present invention provides compounds represented by formula (1), Wherein,

R₁ is hydroxy;
R₂ is hydrogen;
R₃ is selected from methyl, ethyl, propyl, and butyl;
R₄ is formula (4):

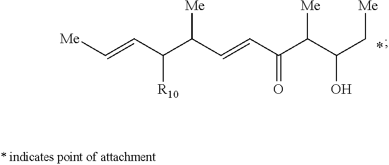

R₁₀ is hydroxy.

In one embodiment, the present invention provides compounds represented by formula (1), wherein,

### R₁ is —O(CO)R₁₃;

R₂ is hydrogen;
R₃ is alkyl;
R₄ is formula (4):

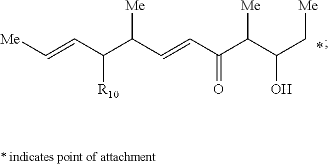

### R₁₀ is —O(CO)R₁₉;

R₁₃ is selected from alkyl, and aryl; and
R₁₉ is selected from alkyl, aralkyl, aryl, and heterocyclyl;
where alkyl is unsubstituted or substituted by one or two of the same or different groups selected from: hydroxy, halogen, amino, hydroxyalkyl, and alkoxy;
aryl is unsubstituted or substituted by one or two of the same or different groups selected from: halogen, hydroxy, amino, alkyl, hydroxyalkyl, and alkoxy;
heterocyclyl is unsubstituted or substituted by one or two of the same or different groups selected from: halogen, hydroxy, amino, alkyl, hydroxyalkyl, and alkoxy.

In one embodiment, the present invention provides compounds represented by formula (1), wherein,

### R₁ is —O(CO)R₁₃;

R₂ is hydrogen;
R₃ is alkyl;
R₄ is formula (4):

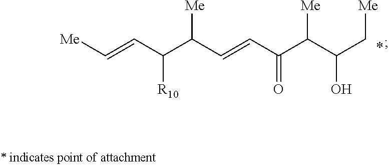

R₁₀ is hydroxy; and
R₁₃ is selected from alkyl, and aryl;
where alkyl is unsubstituted or substituted by one or two of the same or different groups selected from: hydroxy, halogen, amino, hydroxyalkyl, and alkoxy;
aryl is unsubstituted or substituted by one or two of the same or different groups selected from: halogen, hydroxy, amino, alkyl, hydroxyalkyl, and alkoxy.

In one embodiment, the present invention provides compounds represented by formula (1), wherein,

R₁ is halogen;
R₂ is hydrogen;
R₃ is alkyl;
R₄ is formula (4):

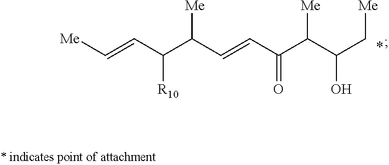

R₁₀ is selected from halogen, hydroxy, alkoxy, —SR₁₄, —NR₁₄R₁₅ and —O(CO)R₁₉;
R₁₄ is selected from hydrogen, alkyl, aralkyl, aryl, and heterocyclyl;
R₁₅ is selected from hydrogen, and alkyl; and
R₁₉ is selected from alkyl, and aryl;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, and alkoxy;
heterocyclyl is unsubstituted or substituted by one or two of the same or different groups selected from: halogen, hydroxy, amino, alkyl, hydroxyalkyl, and alkoxy.

In one embodiment, the present invention provides compounds represented by formula (1), wherein,

R₁ is halogen;
R₂ is hydrogen;
R₃ is alkyl;
R₄ is formula (4):

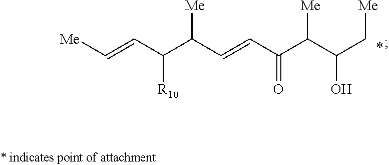

R₁₀ is halogen;
where alkyl is unsubstituted or substituted by one or two of the same or different groups selected from: hydroxy, halogen, amino, hydroxyalkyl, and alkoxy.

In one embodiment, the present invention provides compounds represented by formula (1), wherein,

R₁ is absent and R₂ is ═O;
R₃ is alkyl;
R₄ is formula (4):

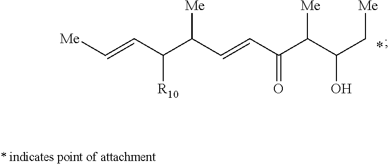

R₁₀ is selected from halogen, hydroxy, alkoxy, —SR₁₄, —NR₁₄R₁₅, and —O(CO)R₁₉;
R₁₄ is selected from hydrogen, alkyl, aralkyl, aryl, and heterocyclyl;
R₁₅ is selected from hydrogen, and alkyl; and
R₁₉ is selected from alkyl, aryl, aralkyl, and heterocyclyl;
where alkyl is unsubstituted or substituted by one or two of the same or different groups selected from: hydroxy, halogen, amino, hydroxyalkyl, and alkoxy;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, and alkoxy;
heterocyclyl is unsubstituted or substituted by one or two of the same or different groups selected from: halogen, hydroxy, amino, alkyl, hydroxyalkyl, and alkoxy.

In one embodiment, the present invention provides compounds represented by formula (1), wherein,

R₁ is absent and R₂ is ═O;
R₃ is methyl;
R₄ is formula (4):

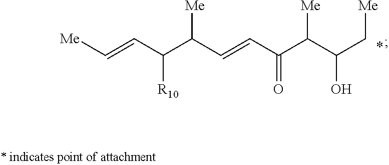

R₁₀ is selected from hydroxy, and alkoxy.

In one embodiment, the present invention provides compounds represented by formula (1), wherein

R₁ is absent and R₂ is ═O;
R₃ is alkyl; and
R₄ is formula (5):

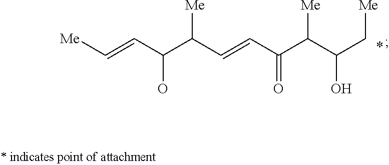

In one embodiment, the present invention provides compounds represented by formula (1), wherein,

R₁ is selected from halogen, hydroxy, and alkoxy;
R₂ is hydrogen;
R₃ is alkyl;
R₄ is formula (8):

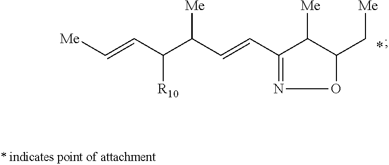

R₁₀ is selected from halogen, and hydroxy;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl.

In one embodiment, the present invention provides compounds represented by formula (1), wherein,

R₁ is selected from hydroxy, and alkoxy;
R₂ is hydrogen;
R₃ is methyl;
R₄ is formula (8):

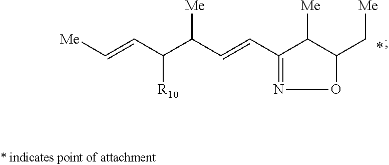

R₁₀ is hydroxy.

In one embodiment, the present invention provides compounds represented by formula (1), wherein,

R₁ is selected from halogen, hydroxy, and alkoxy;
R₂ is hydrogen;
R₃ is alkyl; and
R₄ is formula (9):

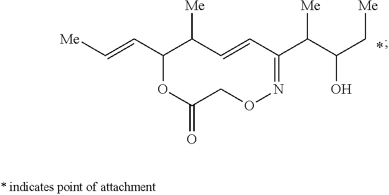

where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl.

The present invention provides compounds of formula (1) (as provided in the above given all embodiments), and all of their stereoisomeric and tautomeric forms and mixtures thereof, in all ratios, and their pharmaceutically acceptable salts, pharmaceutically acceptable solvates, pharmaceutically acceptable polymorphs and prodrugs, for the treatment of an inflammatory disorder mediated by one or more cytokines selected from Tumor Necrosis Factor-alpha (TNF-α), interferon-γ (IFN-γ) and interleukins such as IL-1β, IL-2, IL-6, and IL-8.

The present invention provides compounds of formula (1) (as provided in the above given all embodiments), and all of their stereoisomeric and tautomeric forms and mixtures thereof, in all ratios, and their pharmaceutically acceptable salts, pharmaceutically acceptable solvates, pharmaceutically acceptable polymorphs and prodrugs, for the treatment of an inflammatory disorder mediated by one or more genes selected from CEBPα, CEBPβ, CEBPδ, IL-1β, IL-6, GBP-1, MMP 13, MyD88, BCL2 and cMyc.

The present invention relates to the use of a compound of formula (1) is selected from but not limited to:

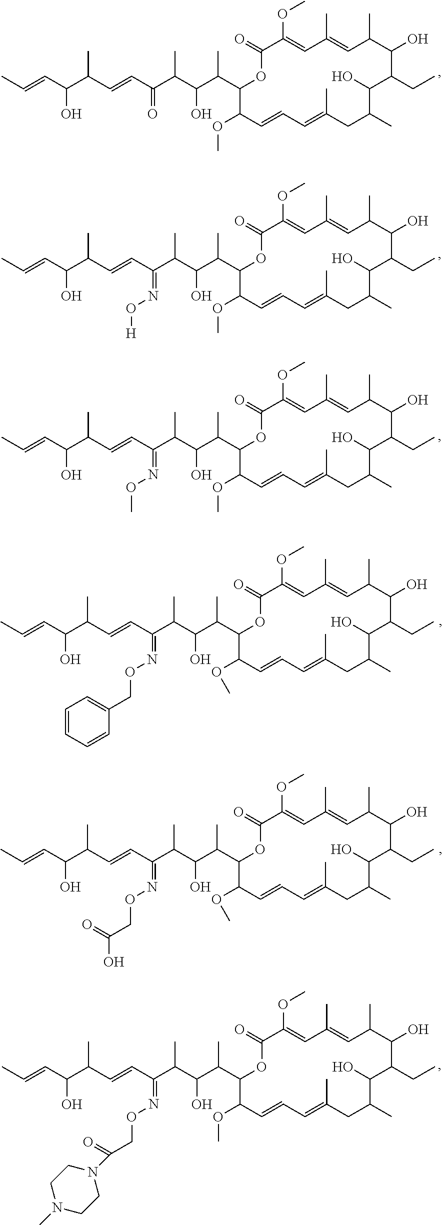

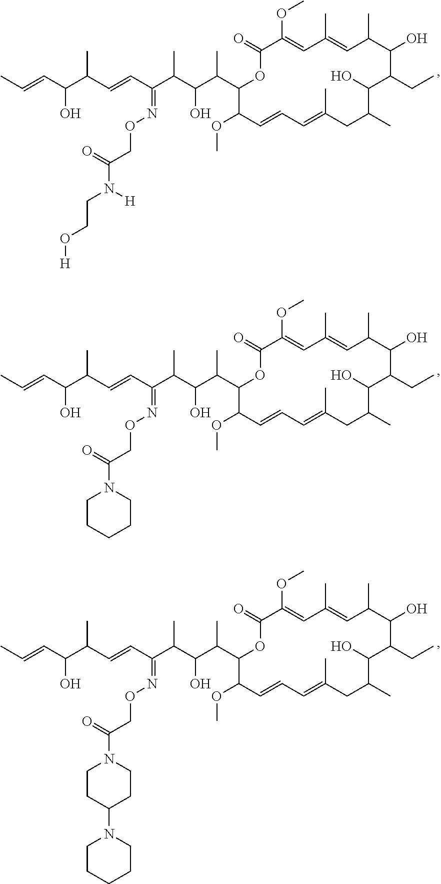

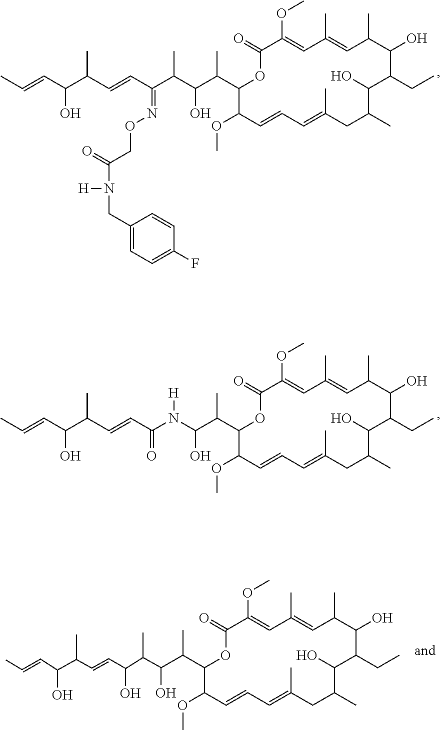

their stereoisomeric and tautomeric forms, pharmaceutically acceptable salts, solvates and prodrugs;
for the treatment of an inflammatory disorder mediated by one or more cytokines selected from Tumor Necrosis Factor-alpha (TNF-α), interferon-γ (IFN-γ) and interleukins such as IL-1(3, IL-2, IL-6, and IL-8.

In another further aspect of the invention, the compound of formula (1) is selected from:

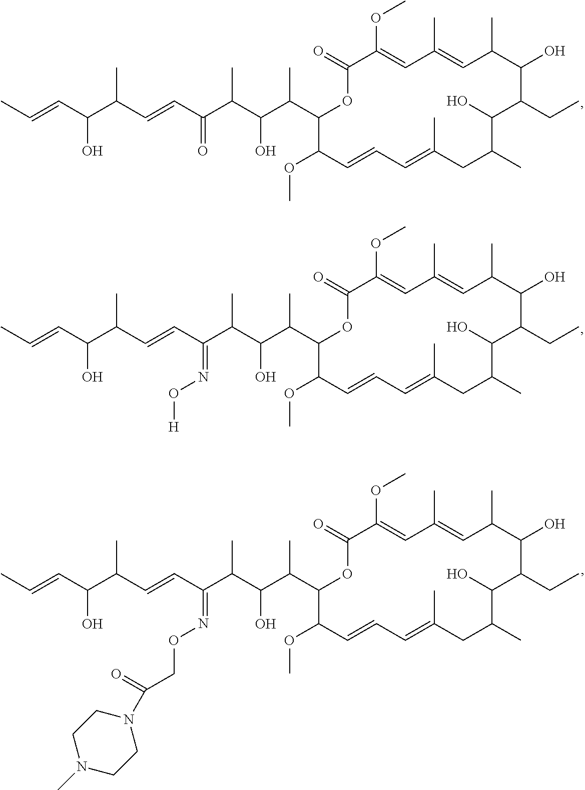

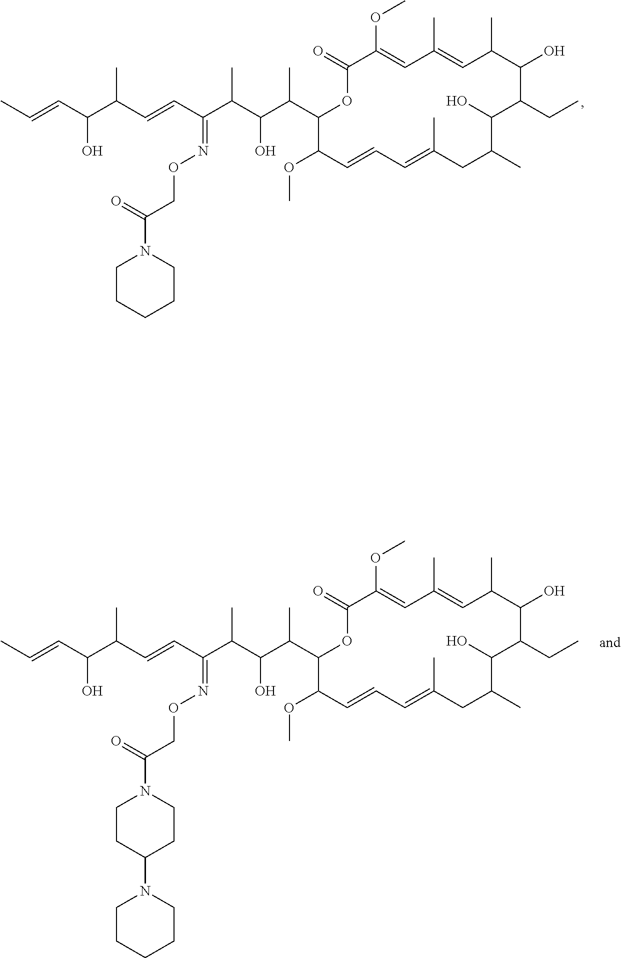

their stereoisomeric and tautomeric forms, pharmaceutically acceptable salts, solvates and prodrugs;
for the treatment of an inflammatory disorder mediated by one or more cytokines selected from Tumor Necrosis Factor-alpha (TNF-α), interferon-γ (IFN-γ) and interleukins such as IL-1β, IL-2, IL-6, and IL-8.

In yet another further aspect of the invention, the compound of formula (1) is:

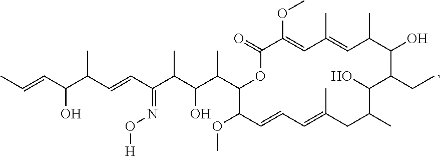

its stereoisomeric and tautomeric forms, pharmaceutically acceptable salts, solvates and prodrugs;
for the treatment of an inflammatory disorder mediated by one or more cytokines selected from Tumor Necrosis Factor-alpha (TNF-α), interferon-γ (IFN-γ) and interleukins such as IL-1β, IL-2, IL-6, and IL-8.

### DETAILED DESCRIPTION OF THE SCHEMES

The compounds of the present invention also include all stereoisomeric forms and mixtures thereof and their pharmaceutically acceptable salts, solvates and polymorphs. Furthermore, all prodrugs and derivatives of the compounds are a subject of the present invention.

According to another aspect of present invention, the compounds of formula (1) can be prepared in a number of ways including using methods well known to the person skilled in the art. Examples of methods to prepare the present compounds are described below and illustrated in Schemes 1 to 4 but are not limited thereto. It will be appreciated by persons skilled in the art that the processes described herein, the order of the synthetic steps employed may be varied and will depend inter alia on factors such as the nature of functional groups present in a particular substrate and the protecting group strategy (if any) to be adopted and will also influence the choice of reagent to be used in the synthetic steps.

The reagents, reactants and intermediates used in the following processes are either isolated from fermentation of microorganisms, are commercially available or can be prepared according to standard literature procedures known in the art or a combination thereof. The starting compounds and the intermediates used for the synthesis of compounds of the present invention, are referred to with general symbols namely (A), (B), (C), (D), (E), (F), (G), (H), (K), (L), (M), (N), (O), (O), (R), (S), (T), and (U). Throughout the process description, the corresponding substituent groups in the various formulae representing starting compounds and intermediates have the same meanings as that for the compounds of formula (1) as described in detailed description.

The processes used in various schemes of the present invention, are referred to with general symbols namely 1a, 1b, 1c, 1d, 1e, 1f, 1 g, 2a, 2b, 2c, 2d, 3a, 3b, 3c, 4a, 4b, 4c, and 4d. Processes for the preparation of compounds of the present invention are set forth in the following schemes:

### Scheme 1

Concanamycin crude (in Scheme 1) is obtained by fermentation of a culture (PM0224355). The whole broth is extracted using a solvent selected from ethyl acetate, chloroform and dichloromethane. Concanamycin crude is isolated by column chromatography and is characterized by spectral comparison (The Journal of Antibiotics, Vol. 45, No. 7, 1108-1116, (1992)).

### Step 1a

Concanamycin crude (in Scheme 1) is subjected to alkaline hydrolysis as per procedure described in reference (Tetrahedron Letters, Vol. 22, No. 39, 3857-60, (1981)), to obtain the compound of formula (1) (wherein R₁ is hydroxy, R₂ is hydrogen, R₃ is methyl, R₄ is formula (4), and R₁₀ is hydroxy; denoted as formula (A) in Scheme 1).

### Step 1b

Compound of formula (1) (wherein R₁ is hydroxy, R₂ is hydrogen, R₃ is methyl, R₄ is formula (3), R₈ is hydroxy, and R₉ is hydroxy; denoted as formula (B) in Scheme 1) is prepared by reacting compound of formula (A) (wherein R₁ is hydroxy, R₂ is hydrogen, R₃ is methyl, R₄ is formula (4), and R₁₀ is hydroxy) with an amine hydrochloride such as hydroxylamine hydrochloride in presence of a base selected from pyridine, substituted pyridine, triethylamine, diisopropylethylamine, N-methylmorpholine, and N-ethylmorpholine using a solvent selected from methanol, ethanol, propanol, butanol, tetrahydrofuran, dimethylformamide, 1,4-dioxane, and acetonitrile. The reaction mixture is stirred at a temperature in the range of 0° C. to 45° C. in an inert atmosphere such as nitrogen gas, over a time period ranging from 4 h to 16 h.

### Step 1c

Compound of formula (1) (wherein R₁ is hydroxy, R₂ is hydrogen, R₃ is methyl, R₄ is formula (6), and R₁₀ is hydroxy; denoted as formula (C) in Scheme 1) is prepared by reacting compound of formula (B) (wherein R₁ is hydroxy, R₂ is hydrogen, R₃ is methyl, R₄ is formula (3), R₈ is hydroxy, and R₉ is hydroxy) in a solvent selected from acetone, acetonitrile, and 1,4-dioxane with tosyl chloride or 2,4,6-trichloro-1,3,5-triazine (TCT), in presence of a base selected from sodium hydroxide and potassium hydroxide, in an inert atmosphere such as nitrogen at 0° C., for 2 h. The reaction mixture can be further stirred at a temperature in the range of 25° C. to 45° C., in an inert atmosphere such as nitrogen gas, over a time period ranging from 2 h to 8 h.

### Step 1d

Compound of formula (1) (wherein R₁ is hydroxy, R₂ is hydrogen, R₃ is methyl, R₄ is formula (3), R₈ is hydroxy, and R₉ is methoxy or benzyloxy; denoted as formula (D) in Scheme 1) is prepared by reacting compound of formula (A) (wherein R₁ is hydroxy, R₂ is hydrogen, R₃ is methyl, R₄ is formula (4), and R₁₀ is hydroxy) with an amine hydrochloride selected from methoxyamine hydrochloride, and benzyloxy amine hydrochloride in presence of a base selected from pyridine, substituted pyridine, triethylamine, diisopropylethylamine, N-methylmorpholine, and N-ethylmorpholine using a solvent selected from methanol, ethanol, propanol, butanol, tetrahydrofuran, dimethylformamide, dioxane, and acetonitrile. The reaction mixture is stirred at a temperature in the range of 0° C. to 45° C., in an inert atmosphere such as nitrogen gas, over a time period ranging from 4 h to 16 h.

### Step 1e

Compound of formula (1) (wherein R₁ is hydroxy, R₂ is hydrogen and R₃ is methyl, R₄ is formula (3), R₈ is hydroxy, R₉ is —OCH₂COOR₁₇, and R₁₇ is hydrogen; denoted as formula (E) in Scheme1) is prepared by reacting compound of formula (A) (wherein R₁ is hydroxy, R₂ is hydrogen, R₃ is methyl, R₄ is formula (4), and R₁₀ is hydroxy) with amine hydrochloride such as carboxymethylhydroxylamine hemi hydrochloride in presence of a base selected from pyridine, substituted pyridine, triethylamine, diisopropylethylamine, N-methylmorpholine and N-ethylmorpholine using a solvent selected from methanol, ethanol, propanol, butanol, tetrahydrofuran, dimethylformamide, 1,4-dioxane, and acetonitrile. The reaction mixture is stirred at a temperature in the range of 0° C. to 45° C., in an inert atmosphere such as nitrogen gas, over a time period ranging from 4 h to 16 h.

### Step 1f

Compound of formula (1) (wherein R₁ is hydroxy, R₂ is hydrogen, R₃ is methyl, R₄ is formula (3), R₈ is hydroxy, R₉ is —OCH₂COR₁₈, R₁₈ is selected from heterocyclyl and —NHCH₂R₂₀, and R₂₀ is selected from alkyl, and aryl; denoted as formula (F) in Scheme 1) is prepared by dissolving compound of formula (E) (wherein R₁ is hydroxy, R₂ is hydrogen, R₃ is methyl, R₄ is formula (3), R₈ is hydroxy, R₉ is —OCH₂COOR₁₇, and R₁₇ is hydrogen) in a solvent selected from dichloromethane, acetonitrile, chloroform, ethyl acetate, and dimethylformamide, and reacting with a coupling reagent selected from dicyclohexylcarbodiimide, 1-ethyl-3-(3-dimethylaminopropyl)carbodiimide hydrochloride

(EDC HCl), N,N′-diisopropyl carbodi imide (DIC), or O-benzotriazol-1-yl-N,N,N′,N′-tetramethyl uranium hexa fluorophosphate (HBTU),O-benzotriazol-1-yl-N,N,N′,N′-tetramethyluroniumtetrafluoroborate(TBTU), benzotriazol-1-yl-oxytripyrrolidinophosphonium hexafluorophosphate(PyBOP) and N-hydroxy benzotriazole (HOBt). Further, the reaction mixture is treated with an amine such as N-methyl-piperazine, ethanolamine, piperidine, 4-piperidino-piperidine, and 4-fluoro phenylamine. The reaction mixture is stirred at a temperature in the range of 25° C. to 45° C., in an inert atmosphere such as nitrogen gas, over a time period ranging from 4 h to 18 h.

### Step 1 g

Compound of formula (1) (wherein R₁ is hydroxy, R₂ is hydrogen, R₃ is methyl, R₄ is formula (7), R₁₀ is hydroxy, R₁₁ is hydrogen, and R₁₂ is hydroxy; denoted as formula (G) in Scheme 1) is prepared by dissolving compound of formula (A) (wherein R₁ is hydroxy, R₂ is hydrogen, R₃ is methyl, R₄ is formula (4), and R₁₀ is hydroxy) in a solvent selected from tetrahydrofuran, acetonitrile, acetone, methanol and ethanol, and is reacted with a reducing agent such as sodium borohydride, in an inert atmosphere such as nitrogen at 0° C. for 20 min. The reaction mixture is further stirred at a temperature in the range of 25° C. to 45° C., in an inert atmosphere such as nitrogen gas, over a time period ranging from 2 h to 8 h.

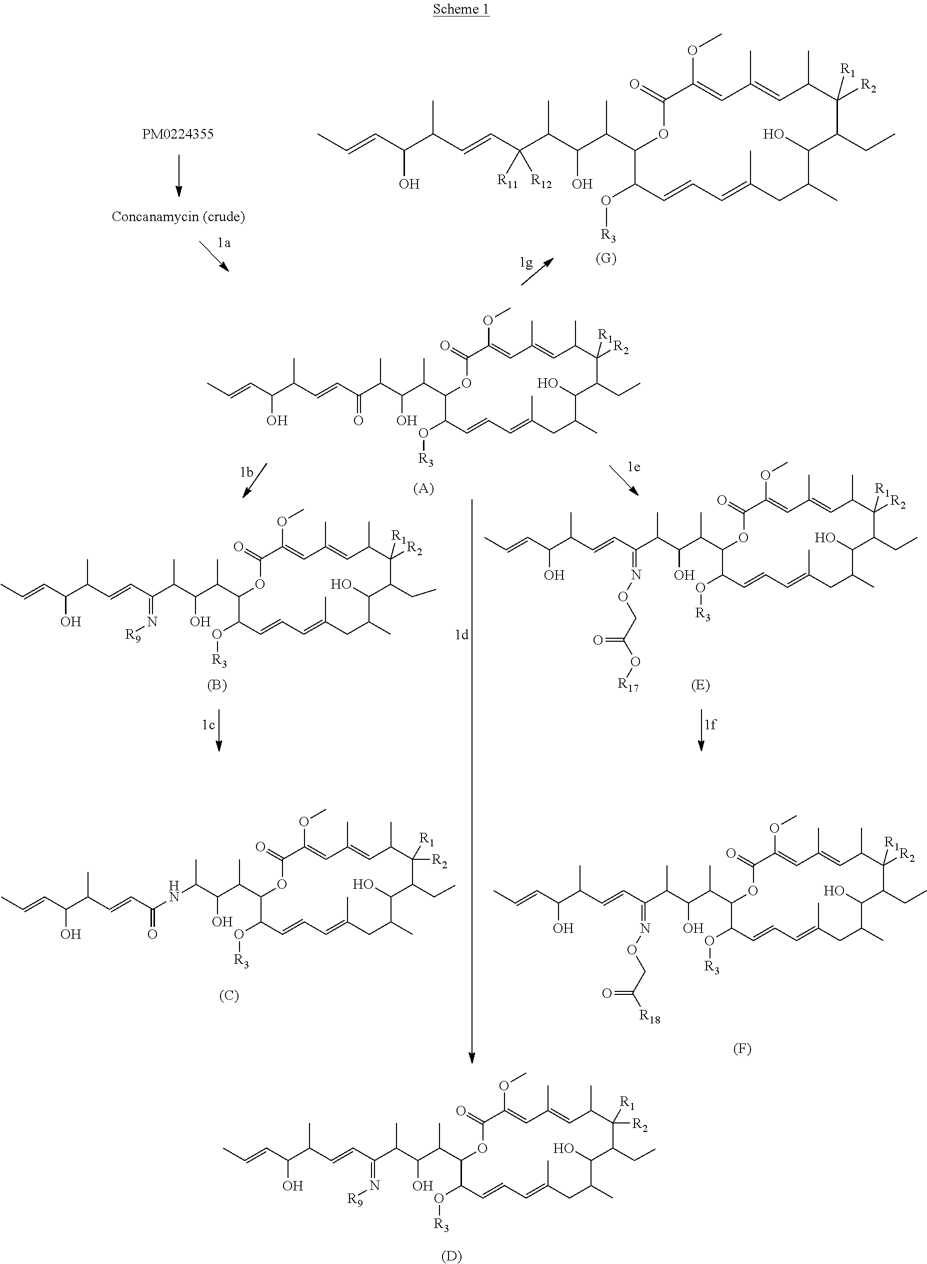

### Step 2a

Compound of formula (1) (wherein R₁ is hydroxy, R₂ is hydrogen, R₃ is methyl, R₄ is formula (8), and R₁₀ is hydroxy; denoted as formula (H), in Scheme 2) is prepared by dissolving compound of formula (B) (wherein R₁ is hydroxy, R₂ is hydrogen, R₃ is methyl; R₄ is formula (3), R₈ is hydroxy, and R₉ is hydroxy; prepared by step 1b, Scheme 1) in a solvent selected from acetone, acetonitrile, 1,4-dioxane, with reagent such as tosyl chloride, in presence of a base selected from sodium hydroxide and potassium hydroxide, in an inert atmosphere such as nitrogen at 0° C., for 2 h. The reaction mixture can be further stirred at a temperature in the range of 25° C. to 45° C., in an inert atmosphere such as nitrogen gas, over a time period ranging from 2 h to 8 h.

### Step 2b

Compound of formula (1) (wherein R₁ is hydroxy, R₂ is hydrogen, R₃ is methyl; and R₄ is formula (9); denoted as formula (K) in Scheme 2) is prepared by dissolving compound of formula (E) (wherein R₁ is hydroxy, R₂ is hydrogen, R₃ is methyl, R₄ is formula (3), R₈ is hydroxy and R₉ is —OCH₂COOR₁₇, R₁₇ is hydrogen; prepared by step 1e, Scheme 1)) in a solvent selected from dichloromethane, acetonitrile, chloroform, ethyl acetate, and dimethylformamide, and reacting with coupling reagent selected from dicyclohexylcarbodiimide, 1-ethyl-3-(3-dimethylaminopropyl)carbodiimide hydrochloride (EDC HCl) and N,N′-diisopropyl carbodiimide (DIC) and catalyst such as 4-dimethylaminopyridine (DMAP). The reaction mixture is stirred at a temperature in the range of 25° C. to 45° C., in an inert atmosphere such as nitrogen gas, over a time period ranging from 4 h to 18 h.

### Step 2c

Compound of formula (1) (wherein R₁ is absent, R₂ is ═O, R₃ is methyl, and R₄ is formula (4), and R₁₀ is hydroxy; denoted as formula (L), in Scheme 2) is prepared by reacting compound of formula (A) (wherein R₁ is hydroxy, R₂ is hydrogen, R₃ is methyl, R₄ is formula (4), and R₁₀ is hydroxy) in a solvent selected from dichloromethane, diethyl ether, and tetrahydrofuran with an oxidizing agent selected from Dess-martin periodinane, pyridinium dichromate, pyridinium chlorochromate, and Swern oxidizing agent in an inert atmosphere such as nitrogen at 0° C. for 2 h. The reaction mixture is further stirred at a temperature in the range of 25° C. to 45° C., under inert atmosphere such as nitrogen gas, over a time period ranging from 2 h to 8 h.

### Step 2d

Compound of formula (1) (wherein R₁ is absent, R₂ is ═O, R₃ is methyl, and R₄ is formula (5); denoted as formula (M), in Scheme 2) is prepared by reacting compound of formula (A) (wherein R₁ is hydroxy, R₂ is hydrogen, R₃ is methyl, R₄ is formula (4) and R₁₀ is hydroxy) in a solvent selected from dichloromethane, diethyl ether, and tetrahydrofuran with an oxidizing agent selected from Dess-martin periodinane, pyridinium dichromate, pyridinium chlorochromate, and Swern oxidizing agent in an inert atmosphere such as nitrogen at 0° C. for 2 h. The reaction mixture is further stirred at a temperature in the range of 25° C. to 45° C., under inert atmosphere such as nitrogen gas, over a time period ranging from 2 h to 8 h.

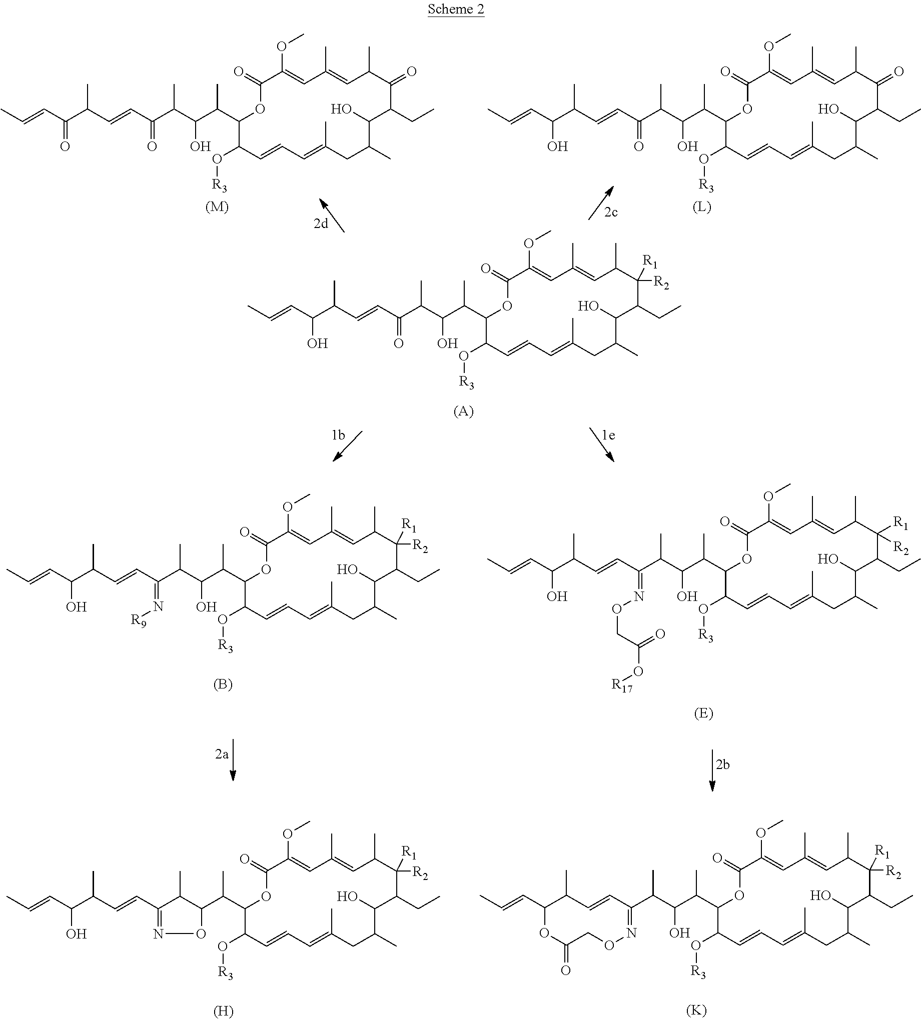

### Scheme 3

### Step 3a

Compound of formula (1) (wherein R₁ is hydroxy, R₂ is hydrogen, R₃ is methyl, R₄ is formula (4), and R₁₀ is —OC(O)R₁₉, R₁₉ is selected from alkyl, aralkyl, aryl, and heterocyclyl; denoted as formula (N), in Scheme 3) is prepared by dissolving compound of formula (A) (wherein R₁ is hydroxy, R₂ is hydrogen, R₃ is methyl, R₄ is formula (4) and R₁₀ is hydroxy) in a solvent selected from dichloromethane, acetonitrile, chloroform, ethyl acetate, and dimethylformamide, and reacting with a coupling reagent selected from dicyclohexylcarbodiimide or EDC HCl or DIC in presence of a catalyst such as DMAP. Further, the reaction mixture is treated with R₁₉—COOH(R₁₉ is selected from alkyl, aralkyl, aryl, and heterocyclyl) is added to the reaction mixture and at a temperature in the range of 25° C. to 45° C., in an inert atmosphere such as nitrogen gas, over a time period ranging from 4 h to 18 h.

### Step 3b

Compound of formula (1) (wherein R₁ is hydroxy, R₂ is hydrogen, R₃ is selected from ethyl, n-propyl, n-butyl and n-pentyl; R₄ is formula (4), and R₁₀ is hydroxy; denoted as formula (O), in Scheme 3) is prepared by dissolving a compound of formula (A) (wherein R₁ is hydroxy, R₂ is hydrogen, R₃ is methyl, R₄ is formula (3), and R₁₀ is hydroxy), in a solvent selected from dichloromethane, acetonitrile, chloroform, ethyl acetate, and dimethylformamide, and reacting with R₃—OH (wherein R₃ is selected from ethyl, n-propyl, n-butyl and n-pentyl) in presence of para-toluene sulphonic acid. The reaction mixture is stirred at a temperature in the range of 25° C. to 45° C., under inert atmosphere such as nitrogen gas, over a time period ranging from 4 h to 18 h.

### Step 3c

Compound of formula (1) (wherein R₁ is hydroxy, R₂ is hydrogen, R₃ is methyl, R₄ is formula (2), R₅ is hydroxy, R₆ is hydrogen, and R₇ is selected from hydrogen or alkyl; denoted as formula (Q), in Scheme 3) is prepared by dissolving a compound of formula (B) (wherein R₁ is hydroxy, R₂ is hydrogen, R₃ is methyl, R₄ is formula (3), R₈ is hydroxy and R₉ is hydroxy; prepared by step 1b, Scheme 1) in a solvent selected from dichloromethane, acetonitrile, chloroform, ethyl acetate, and dimethylformamide, and is reacted with R₇-halide (wherein R₇ is hydrogen or alkyl) in presence of a base selected from triethylamine, diisopropylethylamine, N-methylmorpholine, and N-ethylmorpholine. The reaction mixture is stirred at a temperature in the range of 25° C. to 45° C., in an inert atmosphere such as nitrogen gas, over a time period ranging from 4 h to 18 h.

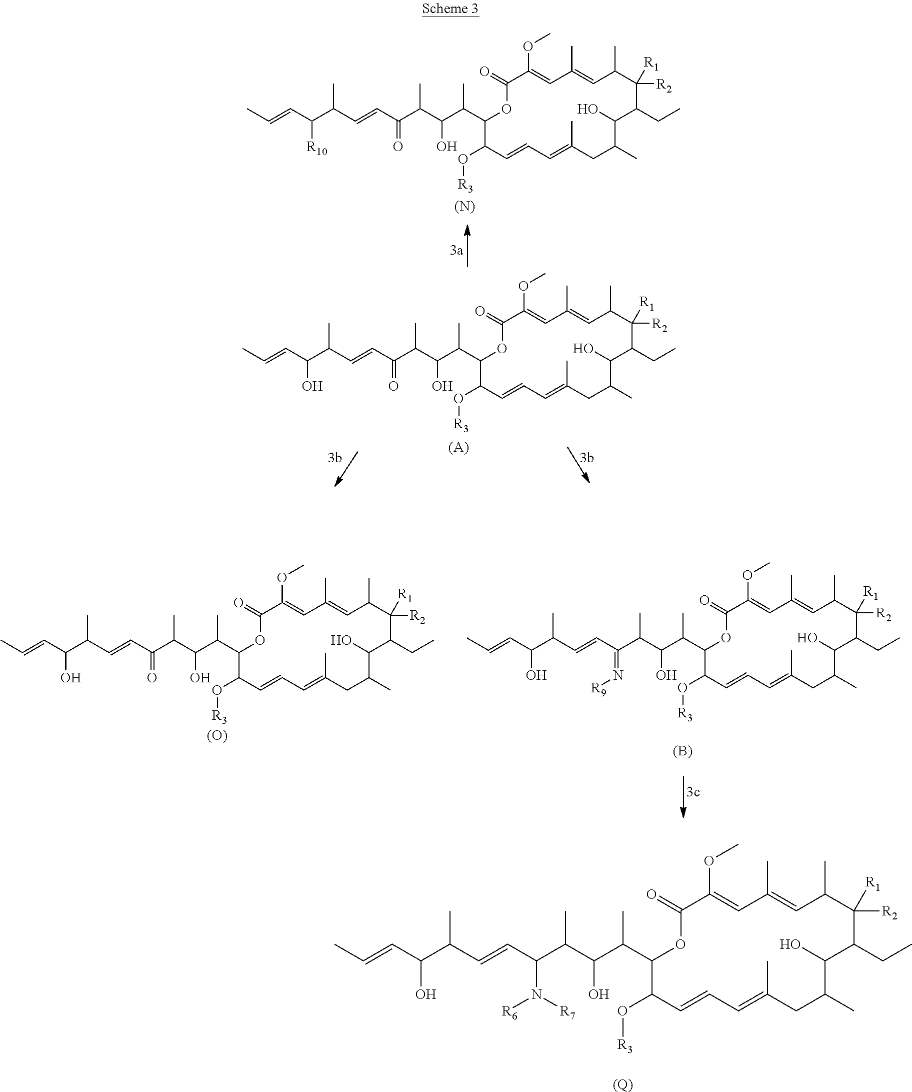

### Scheme 4

### Step 4a

Compound of formula (1) (wherein R₁ is hydroxy, R₂ is hydrogen, R₃ is methyl, R₄ is formula (7), R₁₀ is hydroxy, R₁₁ is halogen and R₁₂ is halogen; denoted as formula (R), in Scheme 4) is prepared by reacting compound of formula (A) (wherein R₁ is hydroxy, R₂ is hydrogen, R₃ is alkyl, R₄ is formula (4), and R₁₀ is hydroxy) with an halogenating agent such as diethylaminosulfur trifluoride (DAST) in a solvent selected from tetrahydrofuran, dimethylformamide, 1,4-dioxane and acetonitrile at a temperature in the range of 0° C. to 45° C., in an inert atmosphere such as nitrogen gas, over a time period ranging from 2 h to 8 h.

### Step 4b

Compound of formula (1) (wherein R₁ is halogen, R₂ is hydrogen, R₃ is methyl, R₄ is formula (4), and R₁₀ is halogen; denoted as formula (S), in Scheme 4) is prepared by reacting compound of formula (A) (wherein R₁ is hydroxy, R₂ is hydrogen, R₃ is methyl, R₄ is formula (4), and R₁₀ is hydroxy) with an halogenating agent such as diethylaminosulfur trifluoride (DAST) in a solvent selected from tetrahydrofuran, dimethylformamide, 1,4-dioxane and acetonitrile at a temperature in the range of 0° C. to 45° C., in an inert atmosphere such as nitrogen gas, over a time period ranging from 2 h to 8 h.

### Step 4c

Compound of formula (1) (wherein R₁ is amino, R₂ is hydrogen, R₃ is methyl, R₄ is formula (4), R₁₀ is —NR₁₄R₁₅, R₁₄ is selected from alkyl, aralkyl, aryl and heterocyclyl and R₁₅ is selected from hydrogen and alkyl; denoted as formula (T), in Scheme 4) is prepared by reacting compound of formula (A) (wherein R₁ is hydroxy, R₂ is hydrogen, R₃ is methyl, R₄ is formula (4), and R₁₀ is hydroxy) with sodium triacetoxyborohydride or sodium cyanoborohydride and amine selected from R₁₄—NH₂ and R₁₄—NH-alkyl (wherein R₁₄ is selected from alkyl, aralkyl, aryl, and heterocyclyl) in a solvent selected from benzene, toluene, tetrahydrofuran, dimethylformamide, 1,4-dioxane and acetonitrile at a temperature in the range of 0° C. to 45° C., in an inert atmosphere such as nitrogen gas, over a time period ranging from 2 h to 8 h.

### Step 4d

Compound of formula (1) (wherein R₁ is SH, R₂ is hydrogen, R₃ is methyl, R₄ is formula (4), and R₁₀ is —SR₁₄, R₁₄ is selected from alkyl, aralkyl, aryl and heterocyclyl; denoted as formula (U), in Scheme 4) is prepared by reacting compound of formula (A) (wherein R₁ is hydroxy, R₂ is hydrogen, R₃ is methyl, R₄ is formula (4), and R₁₀ is hydroxy) with a reducing agent selected from sodium triacetoxyborohydride and sodium cyanoborohydride and R₁₄—SH (wherein R₁₄ is selected from alkyl, aralkyl, aryl and heterocyclyl) in presence of a solvent selected from solvent benzene or toluene, tetrahydrofuran, dimethylformamide, 1,4-dioxane and acetonitrile at a temperature in the range of 0° C. to 45° C., in an inert atmosphere such as nitrogen gas, over a time period ranging from 2 h to 8 h.

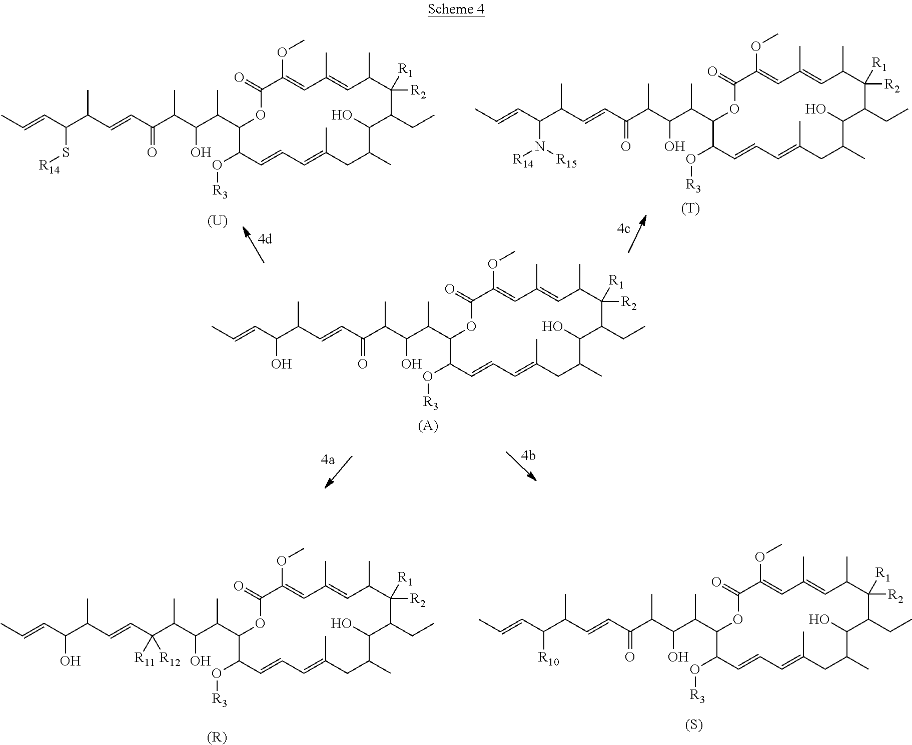

In all the above mentioned schemes 1 to 4, wherever applicable the compounds may be optionally converted into their prodrugs and salts. Additionally the compounds can be separated into individual isomers by techniques well known in the art such as column chromatography.

It will be appreciated by those skilled in the art that the compounds of the present invention can also be utilized in the form of their pharmaceutically acceptable salts or solvates thereof.

With respect to the compounds of formula (1) the present invention also includes all stereoisomeric forms and mixtures thereof in all ratios and their pharmaceutically acceptable salts.

The compounds of the present invention can subsequently be converted into their organic or inorganic salts.

Thus, when the compounds of the present invention represented by the formula (1) contain one or more basic groups, i.e. groups which can be protonated, they can form an addition salt with a suitable inorganic or organic acid. Examples of suitable inorganic acids include: boric acid, perchloric acid, hydrochloric acid, hydrobromic acid, hydrofluoric acid, sulfuric acid, sulfamic acid, phosphoric acid, nitric acid and other inorganic acids known to the person skilled in the art. Examples of suitable organic acids include: acetic acid, propionic acid, succinic acid, glycolic acid, stearic acid, lactic acid, malic acid, tartaric acid, citric acid, ascorbic acid, pamoic acid, maleic acid, hydroxymaleic acid, fumaric acid, phenylacetic acid, glutamic acid, benzoic acid, salicylic acid, sulfanilic acid, 2-acetoxybenzoic acid, toluenesulfonic acid, methanesulfonic acid, benzenesulfonic acid, ethane disulfonic acid, oxalic acid, isethionic acid, ketoglutaric acid, glycerophosphoric acid, aspartic acid, picric acid, lauric acid, palmitic acid, cholic acid, pantothenic acid, alginic acid, naphthoic acid, mandelic acid, tannic acid, camphoric acid and other organic acids known to the person skilled in the art.

The compounds of the present invention represented by the formula (1) contain one or more acidic group they can form an addition salt with a suitable base. For example, such salts of the compounds of the present invention may include their alkali metal salts such as Li, Na, and K salts, or alkaline earth metal salts like Ca, Mg salts, or aluminium salts, or salts with ammonia or salts of organic bases such as lysine, arginine, guanidine, diethanolamine, choline, and tromethamine.

The present invention furthermore includes solvates of the compounds of formula (1), for example hydrates with water and the solvates formed with other solvents of crystallization, such as alcohols, ethers, ethyl acetate, dioxane, dimethylformamide or a lower alkyl ketone such as acetone, or mixtures thereof.

The present invention furthermore includes polymorphs of the compounds of formula (1). Polymorphs may be obtained by heating or melting the compounds of present invention followed by gradual or fast cooling. The presence of polymorphs may be determined by techniques such as IR spectroscopy, solid probe NMR spectroscopy, differential scanning calorimetry, or powder X-ray diffraction.

The present invention also includes prodrugs of the compounds of formula (1), for example esters, amides and other derivatives.

Compounds of the present invention represented by formula (1), are TNF-α inhibitors and find use in therapies for disorders associated with abnormal TNF-α activity, including: inflammatory bowel disease, inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, septic shock, endotoxic shock, atherosclerosis, ischemia-reperfusion injury, coronary heart disease, vasculitis, amyloidosis, multiple sclerosis, sepsis, chronic recurrent uveitis, hepatitis C virus infection, malaria, ulcerative colitis, cachexia, psoriasis, plasmocytoma, endometriosis, Behcet's disease, Wegener's granulomatosis, meningitis, AIDS, HIV infection, autoimmune disease, immune deficiency, common variable immunodeficiency (CVID), chronic graft-versus-host disease, trauma and transplant rejection, adult respiratory distress syndrome, pulmonary fibrosis, recurrent ovarian cancer, lymphoproliferative disease, refractory multiple myeloma, myeloproliferative disorder, diabetes, juvenile diabetes, ankylosing spondylitis, skin delayed-type hypersensitivity disorders, Alzheimer's disease, systemic lupus erythematosus, and allergic asthma.

In certain embodiments, compounds of the invention represented by formula (1), are interleukin (IL-1β, IL-2, IL-6 and IL-8) inhibitors and find use in therapies for disorders associated with abnormal interleukin (IL-1β, IL-2, IL-6 and IL-8) activity, including: rheumatoid arthritis, osteoarthritis and other autoimmune conditions.

In certain embodiments, compounds of the invention represented by formula (1), are IFN-γ inhibitors and find use in therapies for disorders associated with abnormal interleukin (IFN-γ) activity, including: rheumatoid arthritis, osteoarthritis and other autoimmune conditions.

In certain embodiments, compounds of the invention represented by formula (1), down-regulate one or more gene selected from BCL2, CEBPα, CEBPβ, CEBPδ, IL-1β, IL-6, cMyc, GBP-1, MMP13 and MyD88 and find use in therapies for inflammatory disorders including: Burkitt's lymphoma or Peutz-Jeghers syndrome.

According to an embodiment, compounds of the invention represented by formula (1), down-regulate transcriptional targets of CREB such as IL-1β in synovial cells and are useful for the treatment of an inflammatory disorder mediated by CREB pathway.

According to an embodiment, the present invention provides a method for monitoring drug response in a patient with an inflammatory disorder treated with a compound of formula (1), comprising determining the expression of one or more genes selected from CEBPα, CEBPβ, CEBPδ, IL-1β, IL-6, GBP-1, MMP 13, MyD88, BCL2 and cMyc in a test sample from the treated patient and comparing it to the expression of the same one or more genes selected from CEBPα, CEBPβ, CEBPδ, IL-1β, IL-6, GBP-1, MMP 13, MyD88, BCL2 and cMyc in a test sample obtained from the patient before treatment with the compound of formula (1) or in comparison with untreated controls.

In another embodiment, in the method of monitoring drug response, a change of the expression of one or more genes selected from CEBPα, CEBPβ, CEBPδ, IL-1β, IL-6, GBP-1, MMP 13, MyD88, BCL2 and cMyc after treatment is indicative of a drug response.

In another embodiment, in the method of monitoring drug response after the treatment with the compound of formula (1), the expression of one or more genes selected from CEBPα, CEBPβ, CEBPδ, IL-1β, IL-6, GBP-1, MMP 13, MyD88, BCL2 and cMyc is down-regulated.

According to an embodiment, compounds of the invention represented by formula (1), find use in therapies for inflammatory disorders including: inflammatory bowel disease, inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, ulcerative colitis, refractory multiple myeloma, myeloproliferative disorder, psoriasis, common variable immunodeficiency (CVID), skin delayed-type hypersensitivity disorders, and Alzheimer's disease.

According to an embodiment, compounds of the invention represented by formula (1), find use in therapies for inflammatory disorders including: rheumatoid arthritis and ulcerative colitis.

According to an embodiment, compounds of the invention represented by formula (1), find use in the treatment of rheumatoid arthritis.

According to an embodiment, compounds of the invention represented by formula (1), find use in the treatment of ulcerative colitis.

According to an embodiment, compounds of the invention represented by formula (1), find use in the treatment of psoriasis.

According to an embodiment, the present invention provides a method for the treatment of an inflammatory disorder mediated by one or more cytokines selected from Tumor Necrosis Factor-alpha (TNF-α), interferon-γ (IFN-γ), and interleukins such as IL-1β, IL-2, IL-6, and IL-8 by administering to a mammal in need thereof a therapeutically effective amount of one or more compound of formula (1).

According to an embodiment, the present invention provides a method for the treatment of inflammatory disorders associated with abnormal TNF-α activity, including: inflammatory bowel disease, inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, septic shock, endotoxic shock, atherosclerosis, ischemia-reperfusion injury, coronary heart disease, vasculitis, amyloidosis, multiple sclerosis, sepsis, chronic recurrent uveitis, hepatitis C virus infection, malaria, ulcerative colitis, cachexia, psoriasis, plasmocytoma, endometriosis, Behcet's disease, Wegener's granulomatosis, meningitis, AIDS, HIV infection, autoimmune disease, immune deficiency, common variable immunodeficiency (CVID), chronic graft-versus-host disease, trauma and transplant rejection, adult respiratory distress syndrome, pulmonary fibrosis, recurrent ovarian cancer, lymphoproliferative disease, refractory multiple myeloma, myeloproliferative disorder, diabetes, juvenile diabetes, ankylosing spondylitis, skin delayed-type hypersensitivity disorders, Alzheimer's disease, systemic lupus erythematosus, and allergic asthma; by administering to a mammal in need thereof a therapeutically effective amount of one or more compound of formula (1).

According to another embodiment, the present invention provides a method for the treatment of inflammatory disorders associated with abnormal interleukin (IL-1β, IL-2, IL-6 and IL-8) including: rheumatoid arthritis, osteoarthritis and other autoimmune conditions; by administering to a mammal in need thereof a therapeutically effective amount of one or more compound of formula (1).

According to another embodiment, the present invention provides a method for the treatment of inflammatory disorders associated with abnormal interleukin (IFN-γ) activity, including: rheumatoid arthritis, osteoarthritis and other autoimmune conditions; by administering to a mammal in need thereof a therapeutically effective amount of one or more compound of formula (1).

According to an embodiment, the present invention provides a method for the treatment of inflammatory disorders including: inflammatory bowel disease, inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, ulcerative colitis, refractory multiple myeloma, myeloproliferative disorder, psoriasis, common variable immunodeficiency (CVID), skin delayed-type hypersensitivity disorders, Burkitt's lymphoma or Peutz-Jeghers syndrome and Alzheimer's disease; by administering to a mammal in need thereof a therapeutically effective amount of one or more compound of formula (1).

According to an embodiment, the present invention provides a method for the treatment of inflammatory disorders including: rheumatoid arthritis and ulcerative colitis; by administering to a mammal in need thereof a therapeutically effective amount of one or more compound of formula (1).

According to another aspect of the present invention, there are provided pharmaceutical compositions including a therapeutically effective amount of one or more compound of formula (1) as active ingredient and pharmaceutically acceptable carrier, useful in the treatment of an inflammatory disorder mediated by one or more cytokines selected from Tumor Necrosis Factor-alpha (TNF-α), interferon-γ (IFN-γ) and interleukins such as IL-1β, IL-2, IL-6 and IL-8.

According to another aspect of present invention, there are provided methods of treatment of an inflammatory disorders mediated by one or more cytokines selected from Tumor Necrosis Factor-alpha (TNF-α), interferon-γ (IFN-γ) and interleukins such as IL-1β, IL-2, IL-6 and IL-8 using these compositions as described herein above.

According to another aspect of present invention there are provided methods for manufacture of medicaments including one or more compounds of formula (1), which are useful for the treatment of inflammatory disorders mediated by one or more cytokines selected from Tumor Necrosis Factor-alpha (TNF-α), interferon-γ (IFN-γ) and interleukins such as IL-1β, IL-2, IL-6 and IL-8.

The pharmaceutical compositions according to the present invention are prepared in a manner known per se and familiar to one skilled in the art. Pharmaceutically acceptable inert inorganic and/or organic carriers and/or additives can be used in addition to the compound(s) of the formula (1), and/or its physiologically tolerable salts and/or its prodrugs. For the production of pills, tablets, coated tablets and hard gelatin capsules it is possible to use, for example, lactose, corn starch or derivatives thereof, gum arabic, magnesia or glucose, etc. Carriers for soft gelatin capsules and suppositories are, for example, fats, waxes, natural or hardened oils, etc. Suitable carriers for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the various solvents which have been mentioned.

In addition to the active ingredients of the compound of formula (1), and/or its physiologically acceptable salts and/or prodrugs and carrier substances, the pharmaceutical compositions can contain additives such as, for example, fillers, antioxidants, dispersants, emulsifiers, defoamers, flavors, preservatives, solubilizers or colorants. The pharmaceutical compositions of the present invention can also contain two or more compounds of the formula (1) and/or its physiologically tolerable salts and/or their prodrugs. Furthermore, in addition to at least one compound of the formula (1), and/or its physiologically tolerable salts and/or its prodrugs, the pharmaceutical compositions can also contain one or more other therapeutically or prophylactically active ingredients.

The pharmaceutical compositions normally contain about 1 to 99%, for example, about 5 to 70%, or about 10 to about 30% by weight of the compounds of formula (1) or their physiologically tolerable salts or their prodrugs. The amount of the active ingredient of formula (1), and/or its physiologically tolerable salts and/or its prodrugs in the pharmaceutical compositions can, for example, be from about 5 to 500 mg. The dose of the compounds of this invention, which is to be administered, can cover a wide range. The dose to be administered daily is to be selected to suit the desired effect. A dosage of about 0.001 to 100 mg/kg/day of the compound of formula (1) or a prodrug thereof may be administered per day. If required, higher or lower daily doses can also be administered.

Actual dosage levels of the active ingredients in the pharmaceutical compositions of this invention can be varied so as to obtain an amount of the active ingredient, which is effective to achieve the desired therapeutic response for a particular patient, composition, and mode of administration without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular compounds employed, the age, sex, weight, condition, general health and prior medical history of the patient being treated, and like factors well known in the medical arts. The pharmaceutical compositions according to the present invention can be administered orally, for example in the form of pills, tablets, coated tablets, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of solutions or transdermal patches, or in other ways, for example in the form of aerosols or nasal sprays.

It is understood that modifications that do not substantially affect the activity of the various embodiments of this invention are included within the invention disclosed herein.

### EXAMPLES

The following terms/abbreviations/chemical formulae are employed in the examples:
L: Liter
mL: Milliliter
μL: Microliter
g: Gram
mg: Milligram
μg: Microgram
ng: Nanogram
mM: Millimolar
μM: Micromolar
h: Hours
min: Minutes
lpm: Liters per minute
rpm: Revolutions per minute
HPLC: High performance liquid chromatography
TLC: Thin layer chromatography
HOBt: N-Hydroxybenzotriazole
CO₂: Carbon dioxide
NaOH: Sodium hydroxide
KOH: Potassium hydroxide
NaHCO₃: Sodium bicarbonate
Na₂CO₃: Sodium bicarbonate
NaCl: Sodium chloride
HCl: Hydrochloric acid
EtOH: Ethanol
DMSO: Dimethyl sulfoxide
PEG: Polyethylyene glycol
LPS: Lipopolysaccharide
RPMI: Roswell Park Memorial Institute
FBS: Fetal Bovine Serum
PMA: Phorbol myristate acetate
ELISA: Enzyme Linked Immuno Sorbent Assay
IC₅₀: 50% inhibitory concentration
PBS: Phosphate Buffer Saline
DPBS: Dulbecco's Phosphate Buffered Saline
NBF: Normal Buffered Formalin
KRPH buffer: Krebs-Ringer-phosphate buffer
Tris-HCl: 2-Amino-2-(hydroxymethyl)-1,3-opanediolhydrochloride
Th1 cytokines: Thelper 1 cytokines
μCi: Microcurie
PHA: Phytohemagglutinin
CPM: Counts per minute
hPBMC: Human peripheral blood mononuclear cells
EDTA: Ethylenediaminetetraacetic acid
MTS: (3-(4,5-dimethylthiazol-2-yl)-5-(3-carboxymethoxy phenyl)-2-(4-sulfonyl)-2H-tetrazolium)
PMS: Phenazine methosulfonate
FCS: Fetal Calf Serum
RTQ-PCR: Real-time polymerase chain reaction
anti-CD3: anti-cluster of differentiation 3
anti-CD28: anti-cluster of differentiation 28
SDS: sodium dodecyl sulfate
TBS: Tris-buffered saline
HRP: Horse-radish peroxidase
w/v: weight/volume
v/v: volume (of solute) per volume (of solvent)
DSS: dextran sulfate sodium
MAPK: Mitogen activated protein kinase
ATCC: American Type Culture Collection
Hb: Hemoglobin
CMC: carboxymethyl cellulose
DAI: Disease Activity Index
p.o.: oral administration
s.c.: subcutaneous administration
b.i.d: twice daily
Room temperature: 25±5° C.

### Preparation of the Compounds

### Example 1

### Step 1

Isolation of Culture No. PM0224355
a) Composition of the medium (CSPYME agar):

Glucose 15 g, corn steep liquor 5 g, peptone 7.5 g, yeast extract 7.5 g, calcium carbonate 2.0 g, sodium chloride 5 g, demineralized water 1.0 L, final pH (at 25° C.) 7.0.

b) Black soil samples were collected from crop fields near village Hosalingpur, Bellary, Karnataka, India and were transferred into sterile plastic bags. The samples were maintained at 4-8° C.
c) Isolation of actinomycetes from this soil:

Soil (about 1 g) was added to sterile demineralized water (10 mL) and the mixture was heated at 55° C. for 6 min, to enrich actinomyces spores and to limit eubacteria. 100 μL of 10⁻³ dilution of the heated sample was plated on Corn Starch Peptone Yeast Malt Extract (CSPYME) agar (containing amphotericin B, 20 μg/mL) medium by bulk seed method. Visible colonies were picked after 168 h, purified and were maintained on CSPYME slant for use. The culture was assigned culture no. PM0224355.

Culture no. PM0224355 has been deposited with Microbial Type Culture Collection (MTCC), Institute of Microbial Technology, Sector 39-A, Chandigarh-160 036, India, a World Intellectual Property Organization (WIPO) recognized International Depository Authority (IDA) and has been given the accession number MTCC 5340.

### Step 2

Maintenance of Culture No. PM0224355
a) Composition of the medium (ISP2):

Yeast extract 4 g, malt extract 10 g, glucose 4 g, agar 20 g, demineralized water 1.0 L, final pH (at 25° C.) 7.0-7.2.

b) The culture was maintained in glycerol vials at −70° C. for long-term preservation. Periodically its viability was checked using ISP-2 media.

### Example 2

### Fermentation of PM0224355 Culture in Shake Flasks

a) Composition of seed medium:

Glucose 15 g, corn steep liquor 5 g, soybean meal 15 g, calcium carbonate 2 g, sodium chloride 5 g, demineralized water 1.0 L, final pH (at 25° C.) 6.5-7.5.

b) The seed medium (40 mL) was distributed in Erlenmeyer flasks (500 mL) and flasks were autoclaved at 121° C. for 30 min. The flasks were cooled to room temperature and each flask was inoculated with a loopful of the well-grown producing strain (culture no. PM0224355) on the slant and was shaken on a rotary shaker for 70-74 h at 230-250 rpm at 30° C. (±1° C.) to obtain the seed culture.
c) Composition of the production medium:

Glycerol 30 g, glucose 3 g, yeast extract 2 g, sodium chloride 3 g, sodium nitrate 1 g, calcium carbonate 3 g, peptone 3 g, trace salt solution 1 mL/L, demineralized water 1.0 L, final pH (at 25° C.) 6.5-7.5.

d) The production medium (200 mL) was distributed in Erlenmeyer flasks (1000 mL) and flasks were autoclaved at 121° C. for 30 min cooled to 29° C.-30° C. and each flask was seeded with 5 mL of the seed culture (as obtained in example 2 (b)).
e) Fermentation parameters:

Temperature 29° C.-30° C., agitation 230-250 rpm, and harvest time 46-50 h.

The harvest pH of the culture broth was 6.0-7.0.

### Example 3

### Step 1

Extraction of Culture Broth with Ethyl Acetate

The whole broth (1 L) (as obtained in example 2) and was extracted using ethyl acetate (1 L). The organic layer was separated and was concentrated to obtain the crude ethyl acetate extract.

### Step 2

### Purification of Crude Ethyl Acetate Extract

Crude ethyl acetate extract (as obtained in step 1, example 3) was purified by column chromatography (silica gel, methanol in chloroform). The fractions were monitored by TLC (silica gel, chloroform-methanol 9:1, detection: 254 nm). The fraction eluted with 3% methanol in chloroform, was concentrated to obtain a powder. The powder was crystallized using methanol to obtain a white compound.

¹H NMR (DMSO-d₆, 500 MHz): δ 6.62 (dd, 1H), 6.26 (s, 1H), 6.12 (dd, 1H), 5.69 (br d, 1H), 5.55 (br d, 1H), 5.31 (ddq, 1H), 5.12 (dd, 1H), 5.11 (br d, 1H), 4.93 (s, 2H) 4.58 (dd, 1H), 4.25 (t, 1H), 4.1 (dd, 1H) 3.90 (t, 1H), 3.88 (m, 1H) 3.82 (dd, 1H), 3.63 (m, 1H), 3.58 (dd, 1H), 3.54 (s, 3H), 3.35 (dq, 1H), 3.32 (br d, 1H), 3.30 (s, 1H), 2.5 (m, 1H), 2.23 (m, 1H), 2.21 (m, 2H), 2.08 (m, 1H) 2.07 (br 3H), 1.90 (m, 1H), 1.84 (br s, 3H), 1.62 (m, 1H), 1.60 (m, 2H), 1.59 (dq, 1H) 1.57 (d, 3H), 1.37 (d, 3H), 1.28 (m, 1H), 1.23 (m, 2H), 1.12 (d, 3H), 1.10 (d, 3H), 1.07 (d, 3H), 1.01 (d, 3H), 0.89 (t, 3H), 0.82 (d, 3H); MS: m/e 865.

The compound was characterized as concanamycin A by comparison of proton NMR data with the reported data (The Journal of Antibiotics, Vol. 45, No. 7, 1108-1116, (1992)).

The compound obtained in example 3 was used as reference compound.

### Example 4

### Cultivation of the Culture No PM0224355 in Fermenter

### Step 1

### Preparation of Seed Culture in Shake Flasks

a) Composition of the medium:

Glucose 15 g, corn steep liquor 5 g, soybean meal 15 g, calcium carbonate 2 g, sodium chloride 5 g, demineralized water 1.0 L, pH (at 25° C.) 6.5-7.5.

b) The seed medium (200 mL) was distributed in Erlenmeyer flasks (1000 mL) and flasks were autoclaved at 121° C. for 30 min. The flasks were cooled to room temperature and each flask was inoculated with a loopful of the well-grown producing strain (culture no. PM0224355) on the slant and was shaken on a rotary shaker for 70-74 h at 230-250 rpm at 29° C.-30° C. to obtain the seed culture.

### Step 2

### Fermentation

a) Composition of the production medium:

Glycerol 30 g, glucose 3 g, yeast extract 2 g, sodium chloride 3 g, sodium nitrate 1 g, calcium carbonate 3 g, peptone 3 g, trace salt solution 1 mL/L, demineralized water 1.0 L, pH (at 25° C.) 6.5-7.5.

b) In fermenter (150 L), the above production medium (100 L) along with desmophen (30 mL) as an antifoaming agent was sterilized in situ for 30 min at 121° C., was cooled to 29° C.-30° C. and was seeded with 2.5-3.5 L of the seed culture (as obtained in step 1(b), example 4).
c) Fermentation parameters:

Temperature 29° C.-30° C., agitation 100 rpm, aeration 60 lpm, harvest time 46-50 h. The production of the concanamycin in the fermentation broth was determined by TLC (silica gel, chloroform-methanol 9:1, detection: 254 nm) comparison with reference compound concanamycin A. The harvest pH of the culture broth was 6.0-7.0.

### Example 5

### Isolation and Purification of Culture Broth PM0224355

### Step 1

### Extraction

The whole broth (90 L) (as obtained in step 2 (c), example 4) and was extracted using ethyl acetate (90 L). The organic layer was separated and was concentrated to obtain the crude ethyl acetate extract. Yield: 12 g.

### Step 2

### Purification

Crude ethyl acetate extract (as obtained in step 1, example 5) was purified by column chromatography (silica gel, methanol in chloroform). The fractions were monitored by TLC (silica gel, chloroform-methanol 9:1, detection: 254 nm) using concanamycin A as a reference standard. The fraction which was eluted with 3% methanol in chloroform, was concentrated to obtain extract enriched with concanamycins (5 g). The extract enriched with concanamycins was dissolved in methanol, kept at 4° C. for 10-12 h, and was filtered to obtain a powder (Yield: 0.6 g) which was identified as containing mixture of concanamycin A and concanamycin C by LCMS (molecular weight 865 and 822). This is referred to as concanamycin crude.

### Example 6

### (3Z,5E,13E,15E)-18-((6E,10E)-3,9-Dihydroxy-4,8-dimethyl-5-oxododeca-6,10-dien-2-yl)-9-ethyl-8,10-dihydroxy-3,17-dimethoxy-5,7,11,13-tetramethyloxacyclooctadeca-3,5,13,15-tetraen-2-one

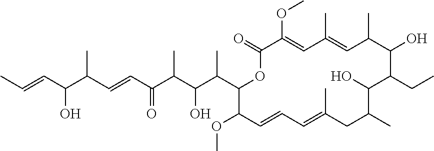

NaOH solution in methanol (0.03M) was added to the powder (100 mg) (as obtained in step 2, example 5) at 10° C. and the mixture was stirred for 20 min. The reaction mixture was neutralized using HCl (1 N) and was extracted with ethyl acetate (3×10 mL). The organic layer was washed with water, dried over sodium sulphate and was concentrated. The crude product was purified by column chromatography (silica gel, 30% ethyl acetate in petroleum ether) to obtain the title compound. Yield: 55 mg.

HPLC: 95% pure [RP-18 (4 mm×250 mm) column, 2-100% gradient of acetonitrile in water over 35 min at 25° C., detection: 220 nm]; MS: m/e 674;

¹H NMR (DMSO-d₆, 500 MHz): δ 6.77 (dd, 1H), 6.6 (dd, 1H), 6.27 (s, 1H), 6.12 (dd, 1H), 5.67 (br. d, 1H), 5.64 (br. d, 1H), 5.49 (dd, 1H), 5.38 (dd. q, 1H), 5.12 (dd, 1H), 5.10 (dq, 1H), 3.89 (t, 1H), 3.80 (dd, 1H), 3.64 (dd, 1H), 3.53 (s, 3H), 3.48 (t, 1H), 3.23 (s, 3H), 2.97 (br. d, 1H), 2.92 (d, 1H), 2.58 (m, 1H), 2.43 (m, 1H), 2.31 (m, 1H), 1.99 (s, 3H), 1.9 (br. s, 3H), 1.88 (m, 1H), 1.86 (m, 2H), 1.63 (dd, 3H), 1.45 (m, 1H), 1.23 (m, 2H), 1.15 (d, 3H), 1.01 (d, 3H), 1.00 (d, 3H) 0.92 (d, 3H), 0.91 (t, 3H), 0.89 (d, 3H).

¹³CNMR (DMSO-d₆, 125 MHz): δ 200.2, 162.4, 147.4, 140.7, 140.4, 140.2, 139.4, 131.7, 131.5, 128.6, 126.6, 125.5, 124.1, 121.3, 80.9, 76.8, 73.3, 72.9, 70.8, 70.0, 58.3, 57.3, 44.8, 44.1, 41.9, 40.9, 37.8, 34.1, 33.3, 20.8, 20.6, 19.2, 16.1, 14.9, 13.8, 12.7, 10.5, 9.0, 7.9.

The compound was characterized as: (3Z,5E,13E,15E)-18-((6E,10E)-3,9-Dihydroxy-4,8-dimethyl-5-oxododeca-6,10-dien-2-yl)-9-ethyl-8,10-dihydroxy-3,17-dimethoxy-5,7,11,13-tetramethyloxacyclooctadeca-3,5,13,15-tetraen-2-one, by comparison of proton NMR data with the reported data (Tetrahedron letters, Vol. 22, No. 39, 3857-60, (1981)).

### Example 7

### (3Z,5E,13E,15E)-18-((5Z,6E,10E)-3,9-Dihydroxy-5-(hydroxymino)-4,8-dimethyl dodeca-6,10-dien-2-yl)-9-ethyl-8,10-dihydroxy-3,17-dimethoxy-5,7,11,13-tetramethyl oxacyclooctadeca-3,5,13,15-tetraen-2-one

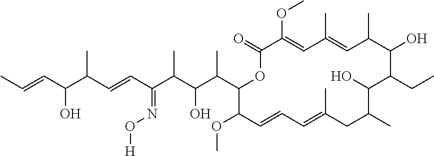

The compound of example 6 (12 mg) was dissolved in the mixture of pyridine (1 mL) and ethanol (1 mL) and was reacted with hydroxylamine hydrochloride (3.17 mg) under nitrogen at 25° C. for 4 h. Water was added to the reaction mixture and the reaction mixture was extracted with ethyl acetate (3×5 mL). The organic layer was washed with water, dried over sodium sulphate and was concentrated. The crude product was purified by column chromatography (silica gel, 40% ethyl acetate in petroleum ether) to obtain the title compound. Yield: 10 mg. HPLC: 99.2% pure, retention time 25.2 min, [RP-18 (4 mm×250 mm) column, 2-100% gradient of acetonitrile in water over 35 min at 25° C., detection: 220 nm]; MS: m/e 689;

¹H NMR (DMSO-d₆, 500 MHz): [symbol] 10.78 (s, 1H), 6.64 (dd, 1H), 6.54 (dd, 1H), 6.27 (s, 1H), 6.05 (dd, 1H), 5.67 (br d, 1H), 5.62 (d, 1H), 5.49 (dd, 1H), 5.38 (ddq, 1H), 5.25 (d, 1H), 5.09 (dq, 1H), 3.88 (t, 1H), 3.78 (dd, 1H), 3.64 (dd, 1H), 3.48 (s, 3H), 3.48 (t, 1H), 3.42 (s, 3H), 2.97 (br d, 1H), 2.89 (d, 1H), 2.54 (m, 1H), 2.42 (m, 1H), 2.28 (m, 1H), 2.01 (s, 3H), 1.96 (m, 2H), 1.87 (m, 1H), 1.83 (br s, 3H), 1.62 (dd, 3H), 1.47 (m, 1H), 1.23 (m, 2H), 1.07 (d, 3H), 1.01 (d, 3H), 0.99 (d, 3H), 0.94 (d, 3H), 0.91 (t, 3H), 0.85 (d, 3H).

¹³C NMR (DMSO-d₆, 125 MHz): δ 164.0, 157.0, 142.3, 141.1, 140.0, 133.7, 133.1, 130.4, 129.5, 127.1, 125.6, 122.9, 120.4, 118.5, 82.9, 78.6, 75.0, 74.7, 72.7, 72.4, 59.4, 55.6, 43.7, 40.4, 39.9, 39.4, 36.1, 35.3, 33.2, 22.7, 22.3, 17.9, 17.8, 16.8, 16.3, 14.4, 12.4, 11.9, 10.7.

### Example 7A

The oxime isomers of compound of example 7 were separated on analytical HPLC [silica gel column (250 mm×4 mm) using 2% methanol in chloroform as eluting solvent; 1 ml/min flow rate]. Isomers have retention time of 8.9 mins and 10.2 mins respectively. Both isomers have same molecular weight of 689.

### Example 8

### (3Z,5E,13E,15E)-18-((5E,6E,10E)-3,9-Dihydroxy-5-(methoxyimino)-4,8-dimethyl dodeca-6,10-dien-2-yl)-9-ethyl-8,10-dihydroxy-3,17-dimethoxy-5,7,11,13-tetramethyloxacycloocta deca-3,5,13,15-tetraen-2-one

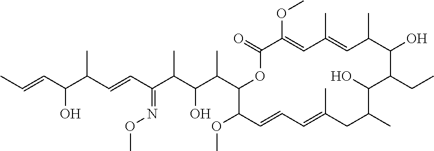

The compound of example 6 (10 mg) was dissolved in the mixture of pyridine (500 μL) and ethanol (500 μL) and was reacted with methoxyamine hydrochloride (6.5 mg) under nitrogen at 25° C. for 4 h. Water was added to the reaction mixture and the reaction mixture was extracted with ethyl acetate (3×5 mL). The organic layer was washed with water, dried over sodium sulphate and was concentrated. The crude product was purified by preparative HPLC [Eurospere-100, C18 column (250 mm×8 mm), mobile phase: acetonitrile-water (1:1 isocratic)] to obtain the title compound. Yield: 6.5 mg; MS: m/e: 703.

### Example 9

### (3Z,5E,13E,15E)-18-((5E,6E,10E)-5-(Benzyloxyimino)-3,9-dihydroxy-4,8-dimethyldodeca-6,10-dien-2-yl)-9-ethyl-8,10-dihydroxy-3,17-dimethoxy-5,7,11,13-tetramethyloxacycloocta deca-3,5,13,15-tetraen-2-one

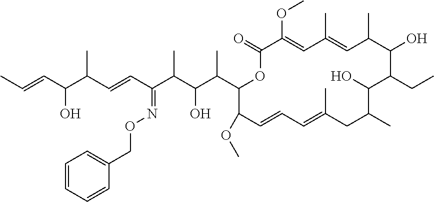

The compound of example 6 (10 mg) was dissolved in the mixture of pyridine (300 μL) and ethanol (700 μL) was reacted with benzyloxy amine hydrochloride (8.4 mg) in pyridine under nitrogen at 25° C. for 4 h. Water was added to the reaction mixture and the reaction mixture was extracted with ethyl acetate (3×5 mL). The organic layer was washed with water, dried over sodium sulphate and was concentrated. The crude product was purified by preparative HPLC [Eurospere-100, C18 column (250 mm×8 mm), mobile phase: acetonitrile-water (1:1 isocratic)] to obtain the title compound. Yield: 7.2 mg; MS: m/e: 779.

### Example 10

### ((Z)-((6E,10E)-2-((4E,6E,14E,16Z)-11-Ethyl-10,12-dihydroxy-3,17-dimethoxy-7,9,13,15-tetramethyl-18-oxooxacyclooctadeca-4,6,14,16-tetraen-2-yl)-3,9-dihydroxy-4,8-dimethyl dodeca-6,10-dien-5-ylidene)aminooxy)acetic acid

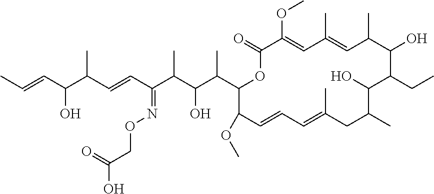

The compound of example 6 (10 mg) was reacted with carboxymethyl hydroxylamine hemihydrochloride (4.8 mg) in pyridine (2 mL) and methanol (4 mL) under nitrogen at 25° C. for 14 h. Water was added to the reaction mixture and the reaction mixture was extracted with ethyl acetate (3×5 mL). The organic layer was washed with water, dried over sodium sulphate was concentrated. The crude product was purified by preparative HPLC [Eurospere-100, C18 column (250 mm×8 mm), mobile phase: acetonitrile-water (1:1 isocratic)] to obtain the title compound. Yield: 7.8 mg; MS: m/e 747.

### Example 11

### (3Z,5E,13E,15E)-18-((5Z,6E,10E)-3,9-Dihydroxy-4,8-dimethyl-5-(2-(4-methyl piperazin-1-yl)-2-oxoethoxyimino)dodeca-6,10-dien-2-yl)-9-ethyl-8,10-dihydroxy-3,17-dimethoxy-5,7,11,13-tetramethyloxacyclooctadeca-3,5,13,15-tetraen-2-one

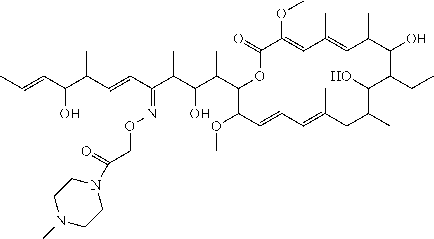

To a solution of compound of example 10 (10 mg) in dichloromethane (2 mL) dicyclohexylcarbodiimide (3 mg), and HOBt (2 mg) were added. After 20 min, N-methyl-piperazine (1.5 mg) was added. The reaction mixture was stirred for 18 h under nitrogen atmosphere. Cold water was added to the reaction mixture, the organic layer was separated. The reaction mixture was extracted with dichloromethane (3×5 mL). The combined organic layer was washed with water (2×5 mL). The organic layer was dried over sodium sulphate, and was concentrated. The crude product was purified by preparative HPLC [Eurospere-100, C18 column (250 mm×8 mm), mobile phase: acetonitrile-water (1:1 isocratic)] to obtain the title compound. Yield: 7.7 mg; ESI-MS: m/e 830 (M+H)⁺.

### Example 12

### 2-((Z)-((6E,10E)-2-((4E,6E,146E,14E,16Z)-1-Ethyl-10,12-dihydroxy-3,17-dimethoxy-7,9,13,15-tetramethyl-18-oxooxacyclooctadeca-4,6,14,16-tetraen-2-yl)-3,9-dihydroxy-4,8-dimethyl dodeca-6,10-dien-5-ylidene)aminooxy)-N-(2-hydroxyethyl)acetamide

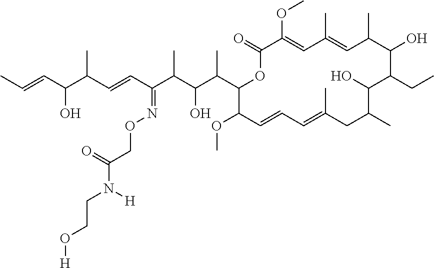

To a solution of compound of example 10 (10 mg) in dichloromethane (2 mL) dicyclohexylcarbodiimide (3 mg), and HOBt (2 mg) were added. After 20 min, ethanol amine (1 mg) was added. The reaction mixture was stirred for 18 h under nitrogen atmosphere. Cold water was added to the reaction mixture, the organic layer was separated. The reaction mixture was extracted with dichloromethane (3×5 mL). The combined organic layer was washed with water (2×5 mL). The organic layer was dried over sodium sulphate, and was concentrated. The crude product was purified by preparative HPLC [Eurospere-100, C18 column (250 mm×8 mm), mobile phase: acetonitrile-water (1:1 isocratic)] to obtain the title compound. Yield: 5.8 mg; ESI-MS: m/e 791 (M+H)⁺.

### Example 13

### (3Z,5E,13E,15E)-18-((5Z,6E,10E)-3,9-Dihydroxy-4,8-dimethyl-5-(2-oxo-2-(piperidin-1-yl)ethoxyimino)dodeca-6,10-dien-2-yl)-9-ethyl-8,10-dihydroxy-3,17-dimethoxy-5,7,11,13-tetramethyloxacyclooctadeca-3,5,13,15-tetraen-2-one

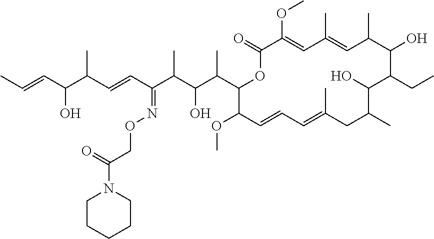

To a solution of compound of example 10 (10 mg) in dichloromethane (2 mL) dicyclohexylcarbodiimide (3 mg), and HOBt (2 mg) were added. After 20 min, piperidine (1.3 mg) was added. The reaction mixture was stirred for 18 h under nitrogen atmosphere. Cold water was added to the reaction mixture, the organic layer was separated. Reaction mixture was extracted with dichloromethane (3×5 mL). The combined organic layer was washed with water (2×5 mL). The organic layer was dried over sodium sulphate, and was concentrated. The crude product was purified by preparative HPLC [Eurospere-100, C18 column (250 mm×8 mm), mobile phase: acetonitrile-water (1:1 isocratic)] to obtain the title compound. Yield: 8.0 mg; ESI-MS: m/e 815 (M+H)⁺.

### Example 14

### (3Z,5E,13E,15E)-18-((5Z,6E,10E)-5-(2-(1,4′-Bipiperidin-1′-yl)-2-oxoethoxyimino)-3,9-dihydroxy-4,8-dimethyldodeca-6,10-dien-2-yl)-9-ethyl-8,10-dihydroxy-3,17-dimethoxy-5,7,11,13-tetramethyloxacyclooctadeca-3,5,13,15-tetraen-2-one

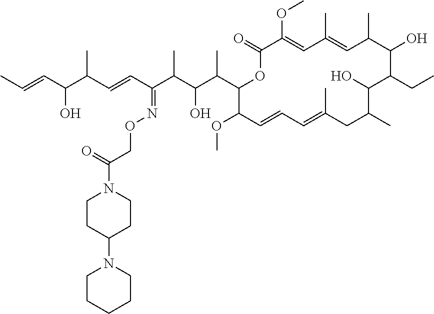

To a solution of compound of example 10 (10 mg) in dichloromethane (2 mL) dicyclohexylcarbodiimide (3 mg), and HOBT (2 mg) were added. After 20 min, 4-piperidino-piperidine 2.5 mg) was added. The reaction mixture was stirred for 18 h under nitrogen atmosphere. Cold water was added to the reaction mixture, the organic layer was separated; Reaction mixture was extracted with dichloromethane (3×5 mL). The combined organic layer was washed with water (2×5 mL). The organic layer was dried over sodium sulphate, and was concentrated. The crude product was purified by preparative HPLC [Eurospere-100, C18 column (250 mm×8 mm), mobile phase: acetonitrile-water (1:1 isocratic)] to obtain the title compound. Yield: 7.5 mg; ESI-MS: m/e 898 (M+H)⁺.

### Example 15

### 2-((Z)-((6E,10E)-2-((4E,6E,146E,14E,16Z)-1-Ethyl-10,12-dihydroxy-3,17-dimethoxy-7,9,13,15-tetramethyl-18-oxooxacyclooctadeca-4,6,14,16-tetraen-2-yl)-3,9-dihydroxy-4,8-dimethyldodeca-6,10-dien-5-ylidene)aminooxy)-N-(4-fluorobenzyl)acetamide

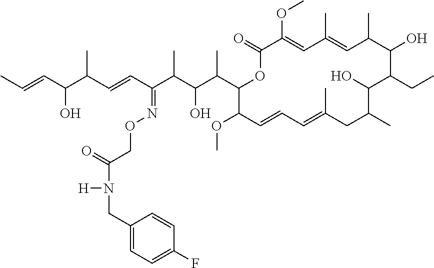

To a solution of compound of example 10 (10 mg) in dichloromethane (2 mL) dicyclohexylcarbodiimide (3 mg), and HOBt (2 mg) were added. After 20 min, 4-fluoro benzyl amine (1.5 mg) was added. The reaction mixture was stirred for 18 h under nitrogen atmosphere. Cold water was added to the reaction mixture, the organic layer was separated. The reaction mixture was extracted with dichloromethane (3×5 mL). The combined organic layer was washed with water (2×5 mL). The organic layer was dried over sodium sulphate, and was concentrated. The crude product was purified by preparative HPLC [Eurospere-100, C18 column (250 mm×8 mm), mobile phase: acetonitrile-water (1:1 isocratic)] to obtain the title compound. Yield: 8.8 mg; ESI-MS: m/e 855 (M+H)⁺.

### Example 16

### (2E,6E)-N-(4-((4E,6E,14E,16Z)-11-Ethyl-10,12-dihydroxy-3,17-dimethoxy-7,9,13,15-tetramethyl-18-oxooxacyclooctadeca-4,6,14,16-tetraen-2-yl)-3-hydroxypentan-2-yl)-5-hydroxy-4-methylocta-2,6-dienamide

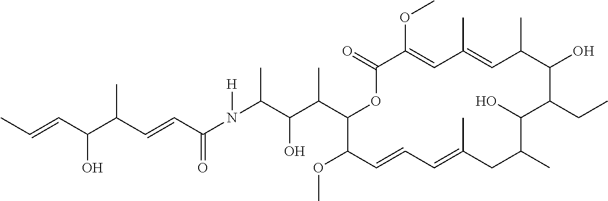

The compound of example 6 (3 mg) was dissolved in acetone (1 mL) and was reacted with KOH (1 mg) and tosyl chloride (1.8 mg) under nitrogen at 0° C. for 2 h. Stirring was continued for 1 h at room temperature. Cold water was added to the reaction mixture and the reaction mixture was extracted with ethyl acetate (3×5 mL). The organic layer was washed with water, dried over sodium sulphate, and was concentrated. The crude product was purified by preparative TLC [silica gel, mobile phase: ethyl acetate-hexane (1:1)] to obtain the title compound. Yield: 0.7 mg. MS: m/e: 689.

### Example 17

### (3Z,5E,13E,15E)-9-Ethyl-8,10-dihydroxy-3,17-dimethoxy-5,7,11,13-tetramethyl-18-((6E,10E)-3,5,9-trihydroxy-4,8-dimethyldodeca-6,10-dien-2-yl)oxacyclooctadeca-3,5,13,15-tetraen-2-one

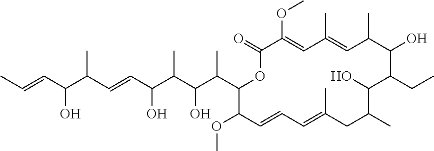

The compound of example 6 (5 mg) was dissolved in tetrahydrofuran (1 mL) and was subjected to reaction with sodium borohydride (0.56 mg) and cerium(III) chloride (CeCl₃) (0.9 mg) under nitrogen at 0° C. for 20 min, then was stirred for 20 min at room temperature. Cold water was added to the reaction mixture and it was extracted with ethyl acetate (3×5 mL). The organic layer was washed with water, dried over sodium sulphate and was concentrated. The crude product was purified by preparative TLC [silica gel, mobile phase: hexane-ethyl acetate (1:1)]. to obtain the title compound. Yield: 2.7 mg; MS: m/e 676.

### Pharmacology

The efficacy of the compounds of formula (1) and formulations, in inhibiting the activity of one or more cytokines selected from TNF-α, interferon-γ (IFN-γ) and interleukins (IL-1β, IL-2, IL-6, and IL-8), was determined by pharmacological assays well known in the art and are described below.

### Example 18

### Screening in LPS Stimulated THP-1 Cells

IL-6 (BD Biosciences, USA) production by LPS (Escherchia coli 0127:B8, Sigma, USA) in THP-1 cells (ATCC number: TIB202) was designed as in reference, Journal of Immunology, 151, 5631-5638, (1993), the disclosure of which is incorporated by reference for the teaching of the assay.

THP-1 cells were cultured in RPMI 1640 culture medium (Gibco BRL, UK) containing 100 U/mL penicillin and 100 mg/mL streptomycin, (100× solution, Sigma, USA) containing 10% FBS (JRH Biosciences, USA). 25,000 cells were seeded per well in 96-well plate (Nunc, USA). The cells were differentiated with PMA (Sigma, USA, prepared as 100 μg/mL stock in RPMI and was diluted to 5 ng/mL). The test compound (prepared as 20 mM stock in DMSO and diluted with DMSO to achieve the following final concentrations in the assay: 100, 10, 1, 0.1, 0.01, 0.001 and 0.0001 μM) or vehicle (0.5% DMSO) were added to the cells and the cells were incubated for 30 min at 37° C. LPS (Sigma, USA, prepared as 1 mg/mL stock in PBS) was added to achieve a final concentration of 1 μg/mL. Plates were incubated at 37° C. for 24 h at 5% CO₂. Supernatants were harvested, and assayed for TNF-α and IL-6 by ELISA as described by the manufacturer (BD Biosciences, USA). Percent inhibition of cytokine release compared to the control was calculated. The IC₅₀ values were calculated by a nonlinear regression method. Results obtained are summarized in Table 1.

TABLE 1
IC₅₀ values in LPS stimulated THP-1 cells

| Sr. | | TNF | IL-6 |
|---|---|---|---|
| No. | Test compound | IC₅₀ (μM) | IC₅₀ (μM) |
| 01 | Compound of example 6 | >100 | 0.10 |
| 02 | Compound of example 7 | 18 | 0.04 |
| 03 | Compound of example 10 | >30 | 2.3 |
| 04 | Compound of example 11 | >30 | 0.3 |
| 05 | Compound of example 13 | >30 | 0.2 |
| 06 | Compound of example 14 | 18 | 0.13 |
| 07 | Compound of example 16 | >100 | 0.03 |

Dexamethasone is used as a standard for this experiment.

### Conclusion:

Representative compounds of the present invention preferably blocked LPS induced IL-6 production in THP-1 cells.

### Example 19

T-Cell Proliferation of Normal hPBMCs

The assay method was designed as in references, The Journal of Immunology, 153, 1-9, (1994), and Clinical and Diagnostic Laboratory Immunology, 7, 687-692, (2000), the disclosure of which is incorporated by reference for the teaching of the assay.

### Step 1

Isolation of hPBMCs

hPBMCs were obtained from healthy donors by centrifugation of heparinized venous blood over Ficoll/Hypaque solution (Histopaque-1077, Sigma, USA). Isolated hPBMCs were suspended in RPMI 1640 culture medium supplemented with 10% FBS and seeded at a density of 50,000 cells/well in a 96-well plate (Nunc, USA). The cells were incubated at 37° C., 5% CO₂ for a period of 24 h. These cells were used for the lymphocyte proliferation as well as cytokine release assay.

### Step 2

### Lymphocyte Proliferation Assay

The plated cells were treated with different concentrations of the test compound (prepared as 20 mM stock in DMSO and diluted with DMSO to achieve the following final concentrations in the assay: 100, 10, 1, 0.1, 0.01, 0.001 and 0.0001 μM) and were incubated for 30 min. The cells were then stimulated with 5 ng/mL PMA (Sigma, USA, prepared as 100 μg/mL stock in RPMI and was diluted to 5 ng/mL) and 5 μg/mL PHA (Sigma, USA, prepared as a 1 mg/mL stock in RPMI). The plates were incubated at 37° C., 5% CO₂ for 48 h. The cells were treated overnight with 0.1 μCi of tritiated thymidine per well (obtained from BARC, India; prepared as stock of 1 mCi/mL) and was diluted in KRPH buffer to 10 μCi/mL and 20 μL and was added per well to obtain a final concentration 0.1 μCi/well) and at the end of 48 h, the anti-proliferative effect of the compound was measured using the following formula:

$$\% Anti \text{-} proliferation = \frac{Control ( CPM ) - Treated ( CPM )}{Control ( CPM )} \times 100$$

Controls consisted of hPBMCs with PHA and PMA (Stimulated), hPBMCs with RPMI (Un-stimulated), hPBMCs with FK506 (positive control, Sigma, USA, prepared as 20 mM stock in DMSO and diluted with DMSO to achieve the following final concentrations in the assay: 100, 10, 1, 0.1, 0.01, 0.001 and 0.0001 μM). Results obtained are summarized in Table 2.

TABLE 2
IC₅₀ values for inhibition of cell proliferation in stimulated hPBMC's and
unstimulated hPBMC's

| | Stimulated hPBMC's | Unstimulated hPBMC's |
|---|---|---|
| Test compound | IC₅₀ (μM) | IC₅₀ (μM) |
| Compound of example | 0.003 | 5.0 |
| 7 | | |
| FK506 | <0.001 | 2.0 |

FK506 was used as a standard to validate the experiment.

### Step 3

### Cytokine Release Assay

The plated cells were treated with different concentrations of the test compound (prepared as mM stock in DMSO and diluted with DMSO to achieve the following final concentrations in the assay: 100, 10, 1, 0.1, 0.01, 0.001, 0.0001, 0.00001 and 0.000001 μM) and incubated for 30 min. The cells were then stimulated with PHA (prepared as 1 mg/mL stock in RPMI 1640 culture medium and used at a final concentration of 5 μg/mL). FK506 was used as a standard. The plates were incubated at 37° C., 5% CO₂ for 48 h. The cytokines in the supernatant collected were detected using ELISA kit (BD biosciences, USA). The cytokines evaluated in the assay were TNF-α, IL-2, IL-6, and IFN-γ. Results obtained are summarized in Table 3. and Table 4

TABLE 3
IC₅₀ values in cytokine release assay of PHA-PMA induced hPBMC's

| | | | | IL-6 | |
|---|---|---|---|---|---|
| Sr. | Test | TNF-α | IL-2 | IC₅₀ | IFN-γ |
| No. | compound | IC₅₀ (μM) | IC₅₀ (μM) | (μM) | IC₅₀ (μM) |
| 01 | Compound of | 0.02 | 0.005 | 0.01 | 0.02 |
| | example 7 | | | | |
| 02 | Compound of | 0.2 | 0.2 | 0.02 | 2 |
| | example 10 | | | | |
| 03 | Compound of | 0.05 | 0.05 | 0.01 | 5.00 |
| | example 11 | | | | |
| 04 | Compound of | 0.02 | 0.2 | 0.5 | 10.00 |
| | example 13 | | | | |
| 05 | Compound of | 0.02 | 0.005 | 0.01 | 0.02 |
| | example 14 | | | | |

FK506 was used as a standard to validate the experiment.

TABLE 4
IC₅₀ values for mitogen stimulated production of cytokines from hPBMC's

| | IL-6 (IC₅₀ | TNF-α (IC₅₀ | IL-2 (IC₅₀ | IFN-γ (IC₅₀ |
|---|---|---|---|---|
| Test compound | μM) | μM) | μM) | μM) |
| Compound of | 0.000007 | 0.07 | 0.007 | 0.1 |
| example 7 | | | | |

### Step 4

Concanavalin-A-Induced IFN-γ Production from hPBMC

Peripheral blood mononuclear cells (hPBMC) were harvested using Ficoll-Hypaque density gradient centrifugation (1.077 g/mL; Sigma Aldrich). hPBMCs were resuspended in RPMI 1640 culture medium (Gibco BRL, Pasley, UK) containing 10% FCS, 100 U/mL penicillin (Sigma Chemical Co. St Louis, Mo.) and 100 mg/mL streptomycin (Sigma Chemical Co. St Louis, Mo.) at 1×10⁶ cells/mL. 1×10⁵ hPBMCs/well were pre-treated with test compound or 0.5% DMSO (carrier control) for 30 min at 37° C. Subsequently, these cells were stimulated with 1 μg/mL concanavalin A (Sigma Chemical Co., St. Louis, Mo.). Following 18 h incubation at 37° C., supernatants were collected and stored at −70° C. until assayed for human IFN-γ by ELISA as described by the manufacturer (OptiEIA ELISA sets, BD BioSciences). In each experiment, cyclosporin (1 μM) was used as a positive control for inhibiting induced IFN-γ production. Results obtained are summarized in Table 5.

TABLE 5
IC₅₀ values for Con-A-induced production of IFN-γ from hPBMCs

| Test compound | IFN-γ (IC₅₀ μM) |
|---|---|
| Compound of example 7 | 0.2 |

### Conclusion:

Representative compounds of the present invention significantly blocked the production of the Th1 cytokines; namely IL-2, IFN-γ and TNF-α and also inhibited IL-6 production.

### Example 20

### Human Monocyte Assay

The assay was designed as in reference, Physiological Research, 52, 593-598, (2003), the disclosure of which is incorporated by reference for the teaching of the assay.

The objective of the assay is to determine whether compounds of the present invention—mediated inhibition of LPS-induced cytokines from monocytic THP-1 cell line translates to physiologically relevant human cells. Accordingly, the effect of compounds of the present invention on LPS—induced cytokine production from freshly isolated human monocytes was ascertained.

Peripheral blood was collected from healthy donors into potassium EDTA vacutainer tubes (BD Biosciences, USA). hPBMCs were isolated using density gradient separation (Histopaque-1077; Sigma, USA) and suspended in assay medium which is RPMI culture medium (Sigma, USA) containing 10% heat inactivated FBS (JRH Biosciences, Australia), 100 U/mL penicillin (Sigma, USA) and 100 mg/mL streptomycin (Sigma, USA). Monocytes in the hPBMCs were counted using a Coulter Counter following which the cells were resuspended at 2×10⁵ monocytes/mL of assay medium. A cell suspension containing 2×10⁴ monocytes was aliquoted per well of a 96-well plate (Nunc, USA). Subsequently, the hPBMCs were incubated for 4-5 h at 37° C., 5% CO₂. During the incubation, the monocytes adhered to the bottom of 96-well plate. Following the incubation, the non-adherent lymphocytes were washed and assay medium was added to adherent monocytes. After 48 h of incubation at 37° C., 5% CO₂, monocytes were pre-treated with various concentrations of test compound (prepared as 20 mM stock in DMSO; 1 μL of 20× concentrated solution of test compound was dissolved in 200 μL cell suspension to achieve a final concentration of 0.03, 0.1, 0.3, 1, 3, 10, 30 and 100 μM) or vehicle (0.5% DMSO) or 10 μM dexamethasone (standard IL-6 and TNF-α inhibitor, Sigma, USA) for 30 min at 37° C., 5% CO₂ and stimulated with 1 μg/mL LPS (Escherchia coli 0111:B4, Sigma, USA). The cells were then incubated for 5 h at 37° C., 5% CO₂ following which supernatants were collected, stored at −70° C. and were assayed later for IL-6, and TNF-α by ELISA (OptiEIA ELISA sets, BD Biosciences, USA). The IC₅₀ values were calculated by a nonlinear regression method using Graph Pad software (Prism 3.03).

In all experiments, a parallel plate was run to ascertain the toxicity of test compounds. A cell proliferation assay kit (Promega Life Sciences, USA), containing MTS tetrazolium salt, was used to assess the viability of the monocytes. Viable cells reduce MTS to form a colored product. The protocol used was as per the manufacturer's instructions and as detailed in the following reference, Am J Physiol Cell Physiol., 285, C813-C822, (2003). A MTS/PMS stock solution was prepared by mixing 2 mL MTS with 100 μL PMS (Sigma, USA) (Stock solution of MTS was prepared as follows: 1 gm of MTS was dissolved in 500 mL of DPBS with calcium and magnesium. Subsequently, the solution was filtered using 0.2 μM filter (Nunc, USA). Aliquots were stored at −20° C. Stock solution of PMS was prepared as follows: 18.4 mg of PMS was dissolved in 20 mL of DPBS with calcium and magnesium. Subsequently, the solution was filtered using 0.2 μM filter sterilized using 0.2 μM syringe filter (Millipore, USA). Aliquots were stored at −20° C. Subsequently, 40 μL of the above mentioned MTS/PMS solution was added to each well of a 96-well plate containing the 2×10⁴ monocytes (resuspended in a volume of 200 μL). After 5 h incubation at 37° C., 5% CO₂, the absorbance of the fluid in each well was determined at 490 nm using the Microwell plate spectrophotometer. The results are summarized in Table 6.

TABLE 6
IC₅₀ values in in vitro Human monocytes assay

| Sr. | | TNF-α | IL-6 |
|---|---|---|---|
| No. | Test compound | IC₅₀ (μM) | IC₅₀ (μM) |
| 01 | Compound of example 6 | 5 | 0.6 |
| 02 | Compound of example 7 | 10 | 0.3 |
| 03 | Compound of example 11 | Not tested | >10 |
| 04 | Compound of example 13 | Not tested | 1.4 |
| 05 | Compound of example 14 | Not tested | 0.15 |

### Conclusion:

Representative compounds of the present invention inhibited LPS-induced production of IL-6 and TNF-α

### Example 21

### Synovial Tissue Assay

The ability of compounds of the present invention to inhibit spontaneous production of cytokines from freshly isolated human synovial tissue cells was designed as in reference, Lancet, 29, 244-247, (1989) the disclosure of which is incorporated by reference for the teaching of the assay.

Synovial tissue was obtained from rheumatoid arthritis patients undergoing knee replacement surgery. The tissue was minced into small pieces and digested in RPMI 1640 culture medium (JRH Biosciences, Australia) containing 100 U/mL penicillin-G, 100 μg/mL streptomycin, 50 ng/mL amphotericin B (Gibco, USA), 1.33 mg/mL collagenase Type I (Worthington Biochemical Corporation, USA), 0.5 μg/mL DNAse Type I (Sigma, USA) and 8.33 U/mL heparin (Biological E. Limited, India) for 3 h at 37° C., 5% CO₂. The digested tissue was filtered through a membrane (mesh size 70 micron; Sigma, USA). Subsequently, the cells were washed 3 times with RPMI 1640 culture medium and resuspended in complete medium (RPMI 1640 culture medium supplemented with 5% FBS and 5% human serum-AB+(Sigma, USA) at a concentration of 1×10⁶ cells/mL. The viability of synovial cells was determined by trypan blue dye exclusion and was uniformly ≧98%. For the experiment, 100 μL of cell suspension was added to the wells of a 96-well culture plate (Nunc, USA). Following cell plating, 100 μL of the culture medium and 1 μL of various concentrations of the test compound (test compound was dissolved in DMSO to obtain a stock solution of 20 mM. 1 μL of 20× concentrated solution of test compound was dissolved in 200 μL cell suspension to achieve a final concentration of 0.03, 0.1, 0.3, 1, 3, 10, 30 and 100 μM in the assay) were added to the cells. The final concentration of DMSO was adjusted to 0.5%. The vehicle (0.5% DMSO) was used as control. The plates were incubated for 16 h at 37° C., 5% CO₂. Subsequently, the supernatants were harvested and stored at −70° C. The amounts of TNF- [symbol] , IL-6 and IL-8 in the supernatants were assayed using OptiEIA ELISA sets (BD BioSciences, USA). The protocol followed was as per manufacturers instructions. The IC₅₀ values were calculated by a nonlinear regression method using the GraphPad software (Prism 3.03).

### Result:

Compound of example 7 inhibited the spontaneous production of IL-6, TNF-α, and IL-8 from freshly isolated synovial tissue cells from rheumatoid arthritis patients. The IC₅₀ of TNF-α, IL-6, and IL-8 inhibition were 19, 0.3 and 1.3 μM respectively. The IC₅₀ for inhibition of TNF-α and IL-6 from synovial tissue cells were comparable to the IC₅₀ values obtained in the human monocyte assay.

### Example 22

hPBMC Membrane—Monocyte Contact Assays

The assay was designed as in reference, Immunology Letters, 117, 114-118, (2008), the disclosure of which is incorporated by reference for the teaching of the assay. Activated T cell contact-mediated monocyte activation, leading to the production of proinflammatory cytokines (e.g., TNF-α, IL-6), contributes to the pathogenesis of chronic inflammatory diseases including rheumatoid arthritis. The objective of this assay is to investigate whether compounds of the present invention inhibit anti-CD3/anti-CD28 activated hPBMC-mediated TNF-α and IL-6 production from monocytes.

### Step 1

### Preparation of Anti-CD3/Anti-CD28 Coated Plates

6-well plates (Nunc, USA) were coated with Goat anti-Mouse IgG, Fc (Chemicon, USA) at a concentration of 16.5 μL/mL in coating buffer (8.4 g/mL NaHCO₃, 3.56 g Na₂CO₃, pH 9.5).

The plates were incubated overnight at 4° C. under sterile conditions. After 24 hours, plates were washed once with sterile PBS (without calcium/magnesium), following which, the plates were incubated with anti-CD3 (5 μg/mL; R&D Systems, USA) and anti-CD28 (1 g/mL; R&D Systems, USA) cocktail in sterile PBS for 3 h. After 3 h, the plates were washed once with PBS, and were used for hPBMC stimulation.

### Step 2

hPBMC Membrane Preparation

The hPBMC membranes were prepared in a manner similar to the preparation of T-cell membranes as described in Immunology Letters, 15, 117(1):114-118, (2008).

Peripheral blood was collected from normal healthy volunteers and hPBMCs were harvested using Ficoll-Hypaque density gradient centrifugation (1.077 g/ml; Sigma, USA). hPBMCs were resuspended in RPMI 1640 culture medium (Gibco BRL, UK) containing 10% FCS (JRH Biosciences, Australia), 100 U/mL penicillin (Sigma, USA) and 100 mg/mL streptomycin (Sigma, USA) at 3.33×10⁶ cells/mL. 5×10⁶ hPBMCs were added per well of a 6-well plate (Nunc, USA) which was uncoated or coated with anti-CD3/anti-CD28.

Subsequently, the hPBMCs in the plate were incubated at 37° C., 5% CO₂ for 24 h.

Following incubation, hPBMCs in separate wells of the 6-well plate were harvested, pooled together, and centrifuged. The supernatants were collected and stored at −70° C. for later analysis for cytokine production as confirmation for anti-CD3/anti-CD28 activation of hPBMCs. The pelleted hPBMCs were washed twice in cold PBS and resuspended in Tris-HCl buffer [PBS containing 50 mM Tris-HCl, pH 7.4; 1 mM EDTA; and protease inhibitor cocktail (Roche, USA)]. The activated/unactivated hPBMCs were broken down by homogenization (Polytron PT 3100 homogenizer) at 10,000 to 12,000 rpm for 1 min, the nucleus fraction was obtained by centrifugation at 4000×g for 15 min, and the supernatant was centrifuged for 45 min at 48,000×g. The pellet of hPBMC membranes was resuspended in lysis buffer (Sigma, USA) and the protein concentration was determined by the method of Bradford (Sigma, USA).

### Step 3

hPBMC Membrane-Monocyte Contact Bioassay:

100 μL monocytes per well at 5×10⁵ cells/mL were added to the 96-well plate (Nunc, USA) and cultured for 48 h at 37° C. Thereafter, supernatants were removed and the cells were further incubated with either unstimulated or anti-CD3/anti-CD28 stimulated hPBMC membranes (0.5-1 μg/mL) or with LPS as a positive control (the stock solution of LPS (1 mg/mL) was prepared in complete medium RPMI 1640 culture medium containing 10% FBS, 100 U/mL penicillin and 100 mg/mL streptomycin). LPS was diluted in complete medium and a 20× solution of LPS was added such that the final concentration of LPS was 1 g/mL in each well containing monocytes. To determine the effect of compounds of the present invention, monocytes were pre-treated with various concentrations of test compound (prepared as 20 mM stock in DMSO. 1 μL of 20× concentrated solution of test compound was dissolved in 200 μL cell suspension to achieve a final concentration of 0.03, 0.1, 0.3, 1, 3, 10, 30 and 100 μM of test compound in the assay) or 0.5% DMSO (vehicle control) for 30 min at 37° C. Stimulated hPBMC membranes were then added to the culture. Supernatants were collected after 24 h and TNF-α and IL-6 production was measured using OptiEIA ELISA sets, (BD BioSciences, USA). The protocol followed was as per manufacturers instructions. In each experiment, supernatants from cultures of monocytes alone without hPBMC cell membranes or hPBMC membranes without monocytes were also collected as negative controls.

### Result:

Compound of example 7 inhibited the activated hPBMC mediated IL-6 production, 66% at 0.3 μM and 100% at 0.5 μM, but did not inhibit TNF-α production from monocytes.

### Conclusion:

Compound 7 of the present invention inhibited the activated hPBMC mediated IL-6 production.

### Example 23

### p38 MAPK Assays

Assays were carried out to select for non inhibitors of p38 MAPK since p38 MAPK inhibitors have demonstrated hepatotoxicity in clinical trials. The method for identifying inhibitors of p38 MAPK was designed as in reference, Journal of Lipid Research, 40, 1911-1919, (1999), the disclosure of which is incorporated by reference for the teaching of the assay.

Human Jurkat T-cells (ATCC number: TIB-152, clone E6-1, USA) were cultured in culture medium (RPMI 1640 culture medium supplemented with 10% FBS, 100 U/mL penicillin and 100 μg/mL streptomycin) at 37° C., 5% CO₂. Culture medium was changed every 2-3 days and always a day prior to the experiment. On the day of the experiment, Jurkat cells were pre-treated with vehicle or test compound at 3 μM, 10 times the IC₅₀ value for IL-6 inhibition in human monocyte assay, for 1 h at 37° C. Subsequently, the cells were stimulated with anisomycin (10 μg/mL; Sigma, USA) for 30 min SB 203580 (1 PM; Sigma, USA) was used as a standard. The sample preparation of the test compound, anisomycin and SB 203580 is as follows: a stock solution (20 mM) of the test compound was prepared in DMSO. All subsequent dilutions of the compound were performed using DMSO. 1 μL of appropriate concentration of the compound was added to the cell suspension to achieve the desired final concentration in the well.

Following the stimulation, cells were harvested, rapidly washed with ice-cold PBS and lysed with cold Cell Lytic buffer (Sigma, USA) supplemented with complete protease inhibitor cocktail (Roche, USA) and sodium orthovanadate (Sigma, USA). The protein extracts were obtained after centrifugation at 15,000 rpm at 4° C. for 20 min. Aliquots of the resulting extracts were analyzed for their protein content using the Coomassie Plus Protein Assay Reagent (Pierce, USA) as per manufacturer's instructions. In all experiments, equivalent amounts of protein (10 μg) were loaded on SDS/12.5%-polyacrylamide electrophoresis gels and resolved at 150 V for 2 h in a buffered solution (24.9 mM Tris base, 250 mM glycine, 0.1% SDS). After electrophoresis, the proteins were transferred from the gel to a nitrocellulose membrane (Sigma, USA) at 25 V for 45 min in transfer buffer (47.9 mM Tris base, 38.6 mM glycine, 0.037% SDS, 20% methanol; pH 9.2-9.4). Blots were blocked in TBS (20 mM Tris base, 0.9% NaCl; pH 7.4) containing 5% nonfat dry milk (Santa Cruz Biotechnology, USA) for 1 h 15 min at room temperature, and incubated with the primary antibody which was prepared in SuperBlock Blocking Buffer in TBS (TBS prepared using Tris from Sigma, USA) at 4° C. overnight with gentle rocking. Primary antibodies included antibodies against phosphor—p38 MAPK (Calbiochem, USA) and beta-actin (Sigma, USA). Following the incubation, membranes were washed and then probed with HRP-conjugated secondary antibody (Calbiochem, USA). Bands were visualized using chemiluminescent peroxidase substrate (Sigma, USA) and a Kodak Imaging station. Blots were stripped with stripping buffer (50 mM Tris-HCl pH 6.8, 1% SDS and 100 mM beta-mercaptoethanol) for min at 50° C., washed and re-probed with a primary antibody to the housekeeping protein beta-actin as a loading control.

### Result:

Compound of example 7 did not inhibit p38 MAP kinase.

### Conclusion:

Representative compound of the present invention did not inhibit p38 MAP kinase.

### Example 24

### Gene Expression Profile Using RTQ-PCR

The effect of the compound of example 7 was measured in stimulated untreated cells from the monocytic cell line (THP-1), human monocytes and synovial cells from rheumatoid arthritis patient. The effect of the compound in the THP-1 cell line was measured in terms of gene expression and was expressed as fold changes as compared to the cell stimulated control with no drug treatment.

THP-1 cells, human monocytes and synovial cells were treated with compound of example 7 or vehicle (0.5% DMSO). Total RNA isolation using a commercial RNA extraction kit (Qiagen Corporation, Germany) The first-strand cDNA was synthesized from total RNA using first strand cDNA synthesis kit from Invitrogen Corporation (California, USA). This was followed by real time quantitative polymerase chain reaction (RTQ PCR) using gene specific primers and standard thermal program of initial denaturation at 95° C. for 5 min and cycles of 95° C. for 10 seconds, followed by 60° C. for 30 seconds (Realplex PCR machine from Eppendorf, Germany). Quantitative measurement of products made during PCR cycles was normalized against a housekeeping gene (Actin) and was used to measure the gene expression as fold changes as compared to respective control. The results are summarized in Table 7A and Table 7B

TABLE 7A

| Genes | Gene Name |
|---|---|
| BCL2 | B-cell CLL/lymphoma 2 |
| CEBPα | CCAAT/enhancer binding protein (C/EBP), alpha |
| CEBPβ | CCAAT/enhancer binding protein (C/EBP), beta |
| CEBPδ | CCAAT/enhancer binding protein (C/EBP), delta |
| IL-1β | Interleukin-1 beta |
| IL-6 | Interleukin-6 |
| cMyc | Myelocytomatosis viral oncogene homolog |
| GBP-1 | Guanylate binding protein 1 |
| MMP13 | Matrix metallo protein 13 |
| MyD88 | Myeloid differentiation primary response gene (88) |

TABLE 7B
Gene expression profile for inflammation markers in THP-1 cell line,
human monocytes and synovial cells after exposure to compound of
example 7 at a concentration of 3 μM.

| Genes | THP-1 cells* | Human monocytes** | Synovial cells*** |
|---|---|---|---|
| BCL2 | −1.3 ± 0.8 | −1.7 + 0.4 | Not done |
| CEBPα | −2.1 + 2.2 | −3.9 + 0.4 | −2.4 |
| CEBPβ | −0.2 + 0.5 | −2.2 + 0.3 | 0.4 |
| CEBPδ | −1.8 + 1.0 | −1.4 + 0.4 | −1.1 |
| IL-1β | −0.6 + 0.7 | −4.0 | −0.3 |
| IL-6 | −1.5 + 1.0 | −1.1 + 1.0 | −4.0 |
| cMyc | −1.1 | −1.9 + 1.7 | −0.5 |
| GBP-1 | −1.6 + 1.0 | Not done | −1.5 |
| MMP13 | −4.1 + 3.5 | −2.2 + 2.0 | −4.0 |
| MyD88 | −0.2 + 0.2 | −0.2 + 1.9 | −0.5 |

*12 h (log fold change ± SE, N = 3)
**3 h (log fold change ± SE, N = 3)
***12 h (log fold change ± SE, N = 1)

Conclusions:

The genes CEBPα, CEBPβ, CEBPδ, IL-1β, IL-6, GBP-1, MMP 13, MyD88, BCL2 and cMyc showed down-regulation in response to the treatment with compound of example 7.

### In Vivo Studies

Animals used in the experiments were housed and cared for, in accordance with the Guidelines in force published by CPCSEA (Committee for the Purpose of Control and Supervision of Experiments on Animals), Tamil Nadu, India. Procedures using laboratory animals were approved by the IAEC (Institutional Animal Ethics Committee) of Piramal Life Sciences Limited, Goregaon, Mumbai, India.

### Example 25

### DSS Induced Murine Model of Acute Colitis

The assay was designed as in references, Laboratory Investigation, 80, 1541, (2000), and Faseb Journal, 19:792, (2005), the disclosure of which is incorporated by reference for the teaching of the assay.

### Step 1

### Induction of Colitis:

Normal, in-house bred female C57BL mice weighing 20-24 μg, 8-10 weeks old, were used. The animals were housed in individually ventilated cages, three per cage, throughout the experimental period.

Experimental colitis was induced in mice by replacing drinking water with 3% (w/v) DSS (molecular weight 36,000-50,000, MP Biomedicals Inc., USA) solution. This solution was prepared in water, freshly every alternate day and was made available to the experimental animals ad libitum, from day 0 to day 10. A batch of six naïve animals received water instead of DSS during this period.

### Step 2

### Treatment:

The animals were weighed every day and the record of body weights was maintained. The suspension of test compound (was prepared at a concentration of 0.05 mg/mL, in 0.5% (w/v) CMC after mixing with minimum quantity of Tween 80 necessary to wet the compound, and was administered orally, daily once to the animals at a volume of 10 mL/kg (dose=0.5 mg/kg). This treatment was initiated on day 6 and continued up to day 10. During this period, DSS control animals and naïve animals received CMC (mixed in the same proportion with 100 μL of Tween 80) at a dose of 10 mL/kg, daily once.

### Step 3

### Terminal Sacrifice:

On day 11, the animals were sacrificed, blood was collected in heparinized tubes and the following parameters were studied;
1. Rectal bleeding/blood in faeces
2. Fecal consistency
3. Bleeding in colon
4. Colon weight
5. Colon length
6. Change in body weight on day 11 from that of day 0
7. Blood hemoglobin concentration

The data are represented in the following tables (Table 8, Table 9 and Table 10) as quantifiable parameters, descriptive parameters and actual scores. The descriptive parameters are represented by DAI. DAI is a research tool used to quantify the symptoms of the colitic animals. DAI is used in order to define response of the treatment or remission of the disease. In order to achieve this, various factors are studied. Some of these factors are quantifiable (e.g. change in body weight during the experimental period, colon length, blood hemoglobin concentration) and hence can be directly used to assess the beneficial effect of the treatment; others are just descriptive (e.g. blood in colon, rectal bleeding, fecal consistency) and are scored according to the severity of the disease. DAI is the sum of the scores of all factors. Results are summarized in Tables 8, 9 and 10.

TABLE 8
Quantifiable parameters

| | Change in | | Colon | Colon weight | Colon weight |
|---|---|---|---|---|---|
| Treatment | body weight | Hbg % | length | mg | mg per cm |
| Control, naive | +0.87 ± 0.23 | 13.28 ± 0.08 | 8.43 ± 0.20 | 265.2 ± 6.45 | 31.5 ± 1.6 |
| Control, DSS | −0.7 ± 0.87 | 7.64 ± 1.39 | 6.27 ± 0.31 | 297 ± 15.60 | 47.5 ± 1.4 |
| DSS + | +0.57 ± 0.28 | 10.77 ± 0.48 | 7.33 ± 0.30 | 273.8 ± 8.93 | 37.6 ± 1.8 |
| Compound of | | | | | |
| example 7 | | | | | |

TABLE 9
Descriptive parameters
Method of scoring

| Feature scored | Score | Description |
|---|---|---|
| Rectal bleeding | 0 | Absent |
| | 1 | Slightly present |
| | 2 | Present |
| | 3 | Profuse |
| Fecal consistency | 0 | Normal |
| | 1 | Slightly loose |
| | 2 | Loose |
| | 3 | Diarrhea |
| Blood in colon | 0 | Absent |
| | 1 | Slightly present |
| | 2 | Present |
| | 3 | Profuse |

DAI = Sum of the scores of all features

TABLE 10
Actual Scores

| | Rectal | Fecal | Blood | Disease |
|---|---|---|---|---|
| Treatment | bleeding | consistency | in colon | activity index |
| Naive | 0 | 0 | 0 | 0 |
| DSS Control | 1.37 ± 0.29 | 2.0 ± 0.0 | 0.66 ± 0.42 | 4.0 ± 0.53 |
| Compound | 0.18 ± 0.15 | 1.0 ± 0.50 | 0.61 ± 0.30 | 1.67 ± 0.47 |
| of example 7 | | | | |

### Conclusion:

Compound of example 7, when administered orally at a dose of 0.5 mg/kg to the experimental animals (mice) with DSS induced colitis, reduces the severity of colitis.

### Step 4

### Histopathology

On the last day of compound treatment (compound of example 7, 0.5 mg/kg, p.o., b.i.d) the animals were euthanized humanely, blood samples were collected and the colon was excised. Anterior part of the colon was washed with normal saline to remove fecal material and then fixed in 10% NBF (Neutral buffered formalin). After 10 days of fixation the colon specimens were trimmed and processed overnight using automated tissue processor. Following day the specimens were blocked in paraffin and exposed to cold shock overnight at −18° C. Sections (5μ) were made from cross section of the colonic lumen and stained with routine Hematoxyllin (Sigma, USA) & Eosin (Loba Chemie, India) and mounted permanently. Slides were dried for 24 h and then graded histologically. The results were expressed as histological scores. Histological analysis was based on various parameters like presence of inflammatory cells, erosions, crypt destruction, edema and overall architectural changes graded on a score of 0 to 3, wherein 0 corresponds to absence, 1 corresponds to changes in 25% of the circumference of the colonic lumen, 2 corresponds to up to 50% and 3 corresponds to more than 50% of colonic circumference getting affected. All the scores were summed up to arrive to a total histological score for each section. Two random sections were graded and mean was calculated.

### Result:

Group mean histological scores were 7.50+0.96 for DSS control and 5.33+0.99 for mice treated with compound of example 7.

### Conclusion:

Compound 7 of the present invention when administered to DSS induced colitic C57BL male mice, confer protection against histological damage.

### Example 26

### Collagen-Induced Arthritis in Mice

The experiment was designed as in reference, J. Experimental Medicine, 162, 637-646 (1985), the disclosure of which is incorporated by reference for the teaching of the experiment.

Male DBA/1J mice with body weight range of 18-22 g, aged 8-10 weeks were immunized with an emulsion equivalent to 200 μg of type II collagen (Elastin products, USA) in Freund's Complete Adjuvant (Sigma, USA), injected intradermally at the base of the tail. The animals were boosted with 200 μg of freshly prepared typeII collagen emulsion emulsified in Freund's Complete Adjuant (Sigma, USA) on day 21. A group of naïve mice was also maintained alongside. Naïve animals are the animals which are neither immunized for induction of arthritis nor do they receive any treatment. This group is maintained to take care of the normal changes in the paw thickness with age.

From day 23, mice were examined daily once for the signs of rheumatoid arthritis, using the articular index and paw thickness as parameters. Articular index scoring was performed employing the following criteria:

### FORELIMBS: SCALE 0-3

0: No redness or swelling
1: Redness, but no swelling
2: Redness and swelling of the paw
3: Redness and severe swelling of the paw
   Hind limbs: Scale 0-4
0: No redness or swelling
1: Redness and mild swelling of paw
2: Redness and moderate swelling of paw and/or swelling of at least one of the digits
3: Redness and moderate/severe swelling of paw, swelling of ankle joint and/or swelling of one or more digits
4: Redness and severe swelling of paw, digits and ankle joint, with joint stiffness Mice with a minimum hind paw score of 2, of even one paw, were inducted into the study. Mice were randomized into the various study groups, each group having at least eight animals, and were administered the vehicle (0.5% CMC, 10 mL/kg p.o. and s.c. twice daily), the test compound (5 mg/kg, p.o. and s.c., twice daily) and standard compound Enbrel (Wyeth Limited, UK), 3 mg/kg, s.c., once daily. The test compound was administered as a suspension in CMC. The requisite quantity of the compound was accurately weighed and was hand-pulverized using pestle-mortar. After mixing with minimum quantity of Tween 80 necessary to wet the compound, requisite quantity of 0.5% CMC solution was added and the compound was ground with CMC till the uniform suspension was obtained. Standard compound ‘Enbrel’ was used as an aqueous solution. The dosing of the compounds was done for 12 continuous days.

The following parameters were observed and recorded daily,
1. Body weight
2. Articular index
3. Paw thickness of hind limbs only, in mm using a tension free calipers
4. Any significant observation regarding the condition of the animals.

On 13^{th} day morning, 1 h after the compound treatment, the animals were sacrificed, blood withdrawn, and plasma collected for drug level analyses. Also, the hind limbs of all the animals were preserved for histopathological evaluations.

### Result:

Compound of example 7 at a dose of 5.0 mg/kg as a CMC suspension, administered subcutaneously twice daily to the mice with collagen induced arthritis, for 12 continuous days, reduced the severity of arthritis. The benefit is equal to that achieved with Enbrel treatment (3 mg/kg, s.c., once daily).

### Histopathology:

Histological score of compound of the section of paws of mice treated with the compound of example 7 (5 mg/kg, s.c., n=10) was 3.6+1.54, Enbrel (3 mg/kg, s.c., n=6) treated mice had a score of 5.8+0.95 and that of vehicle control (n=7) was 15.14+1.0.

### Observations:

The vehicle control group animals showed complete destruction of joint architecture accompanied by severe hyperplasia of synovium and pannus formation. Animals treated with compound of example 7 showed protection against arthritic changes with absence of hyperplasia of synovium.

### Example 27

### Evaluation of Test Compound in Arthritic Mice: Administration by Osmotic Pumps

The experiment was performed in DBA/1J mice, in which arthritis was developed by injection of collagen emulsion, as described in Example 26.

The animals were divided into two groups, viz. a control group and a test compound treated group, having 6 animals each. The clear solution of test compound was prepared in 100% dimethyl sulfoxide (DMSO) and then DMSO concentration was brought down to 25% by addition of appropriate quantities of ethanol and polyethylene glycol 400 (PEG 400), so that the proportion of each solvent in a final solution v/v was 25:15:60::DMSO:EtOH:PEG-400. By this method absolutely clear solution of the compound having a final concentration of 40 mg/mL was obtained. This solution was filtered through 0.2μ filter and was filled in osmotic pumps (Alzet micro-osmotic pump model 1002). The delivery rate of this model of pump is 0.25 μL per hour and it remains functional for 14 days.

These pumps were then implanted sub-cutaneously in the animals of the test compound treated group (240 μg/mouse/24 h). The pumps filled with blank solvent were implanted sub-cutaneously in the animals of control group. Thereafter, their paws were scored for arthritic indices, in addition to measurement of thickness, daily once. After 14 days, the pumps were replaced with freshly filled pumps and the experiment was continued for next 12 days (total 26 days).

### Result:

The compound 7 of present invention, when sub-cutaneously administered in the arthritic animals by means of the osmotic pumps reduces severity of arthritis by the reductions in the arthritic scores and paw thickness, when compared to the control group of animals.

### Conclusion:

Compound 7 of present invention is efficacious in reducing the severity of arthritis when administered sub-cutaneously.

1-26. (canceled)

27. A method for the treatment of an inflammatory disorder, comprising administering to a subject in need thereof a therapeutically effective amount of a compound of the following formula (1), or a stereoisomeric form, or a tautomeric form, or a pharmaceutically acceptable salt thereof, 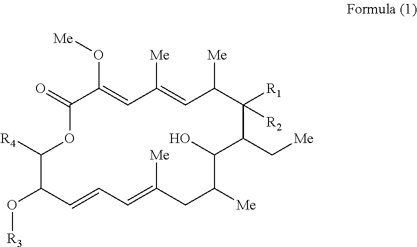
wherein,
R₁ is selected from halogen, hydroxy, alkoxy, —O(CO)R₁₃, —SR₁₄, and —NR₁₄R₁₅;
R₂ is hydrogen; or
optionally R₁ is absent and R₂ is ═O;
R₃ is alkyl;
R₄ is selected from the following formulae: 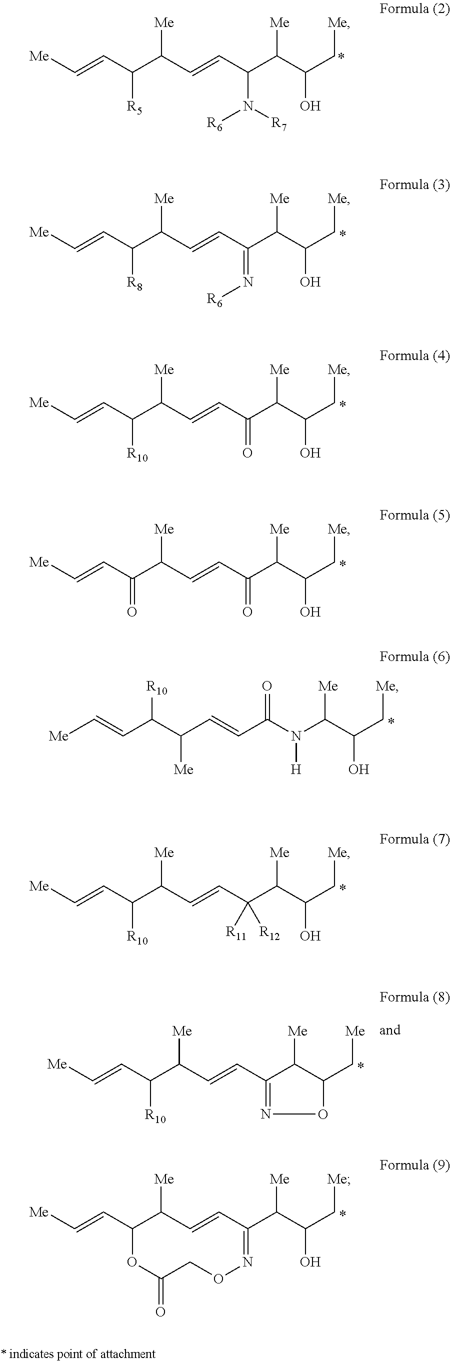
R₅ is selected from hydroxy or alkoxy;
R₆ is selected from hydrogen, hydroxy, alkyl, and alkoxy;
R₇ is selected from hydrogen, alkyl, and —(CO)R₁₆;
R₈ is selected from hydroxy or alkoxy;
R₉ is selected from hydroxy, alkyl, alkoxy, aryl, aralkyl, aryloxy, benzyloxy, heterocyclyl, —O-heterocyclyl, —OCH₂COOR₁₇, and —OCH₂COR₁₈;
R₁₀ is selected from halogen, hydroxy, alkoxy, —SR₁₄, —NR₁₄R₁₅, and —O(CO)R₁₉;
R₁₁ is selected from hydrogen or halogen;
R₁₂ is selected from hydrogen, halogen, and hydroxy;
R₁₃ is selected from alkyl or aryl;
R₁₄ is selected from hydrogen, alkyl, aralkyl, aryl, and heterocyclyl;
R₁₅ is selected from hydrogen or alkyl;
R₁₆ is selected from alkyl or aryl;
R₁₇ is selected from hydrogen, or alkyl;
R₁₈ is selected from alkyl, —NHCH₂R₂₀, aryl, and heterocyclyl;
R₁₉ is selected from alkyl, aralkyl, aryl, and heterocyclyl; and
R₂₀ is selected from hydrogen, alkyl, aryl, and heterocyclyl;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, alkoxy, aryl, and heterocyclyl;
heterocyclyl is unsubstituted or substituted by one or two of the same or different groups selected from: halogen, hydroxy, amino, alkyl, hydroxyalkyl, alkoxy, aryl, and heterocyclyl.

28. The method according to claim 27, wherein in the compound of formula (1)
R₁ is selected from halogen, hydroxy, alkoxy, —O(CO)R₁₃, —SR₁₄, and —NR₁₄R₁₅;
R₂ is hydrogen;
R₃ is alkyl;
R₄ is selected from the following formulae: 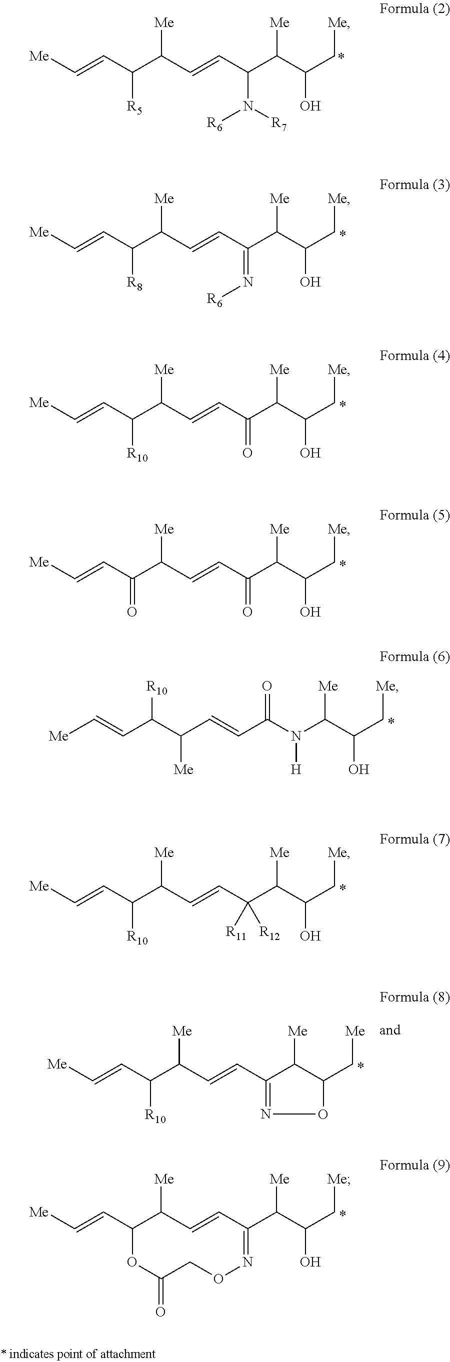
R₅ is selected from hydroxy, or alkoxy;
R₆ is selected from hydrogen, hydroxy, alkyl, and alkoxy;
R₇ is selected from hydrogen, alkyl, and —(CO)R₁₆;
R₈ is selected from hydroxy, or alkoxy;
R₉ is selected from hydroxy, alkyl, alkoxy, aryl, aralkyl, aryloxy, benzyloxy, heterocyclyl, —O-heterocyclyl, —OCH₂COOR₁₇, and —OCH₂COR₁₈;
R₁₀ is selected from halogen, hydroxy, alkoxy, —SR₁₄, —NR₁₄R₁₅, and —O(CO)R₁₉;
R₁₁ is selected from hydrogen, or halogen;
R₁₂ is selected from hydrogen, halogen, and hydroxy;
R₁₃ is selected from alkyl, or aryl;
R₁₄ is selected from hydrogen, alkyl, aralkyl, aryl, and heterocyclyl;
R₁₅ is selected from hydrogen, or alkyl;
R₁₆ is selected from alkyl, or aryl;
R₁₇ is selected from hydrogen, or alkyl;
R₁₈ is selected from alkyl, —NHCH₂R₂₀, aryl, and heterocyclyl;
R₁₉ is selected from alkyl, aralkyl, aryl, and heterocyclyl; and
R₂₀ is selected from hydrogen, alkyl, aryl, and heterocyclyl;
where alkyl is unsubstituted or substituted by one or two of the same or different groups selected from: hydroxy, halogen, amino, hydroxyalkyl, alkoxy, aryl, aryloxy, and heterocyclyl;
alkoxy is unsubstituted or substituted by one or two of the same or different groups selected from: halogen, hydroxy, alkyl, and hydroxyalkyl;
aryl is unsubstituted or substituted by one or two of the same or different groups selected from: halogen, hydroxy, amino, alkyl, hydroxyalkyl, alkoxy, aryl, and heterocyclyl;
heterocyclyl is unsubstituted or substituted by one or two of the same or different groups selected from: halogen, hydroxy, amino, alkyl, hydroxyalkyl, alkoxy, aryl, and heterocyclyl.

29. The method according to claim 27, wherein the compound of formula (1) is selected from: 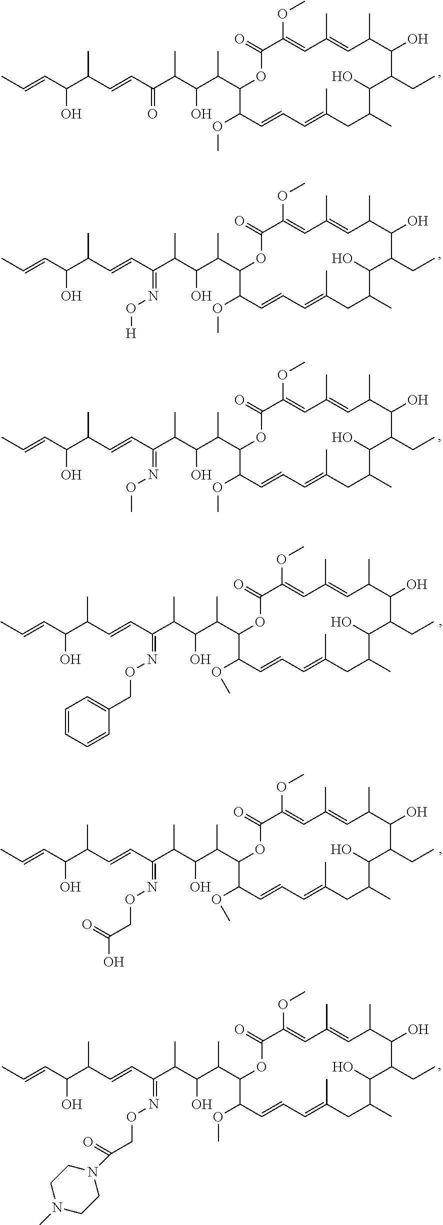 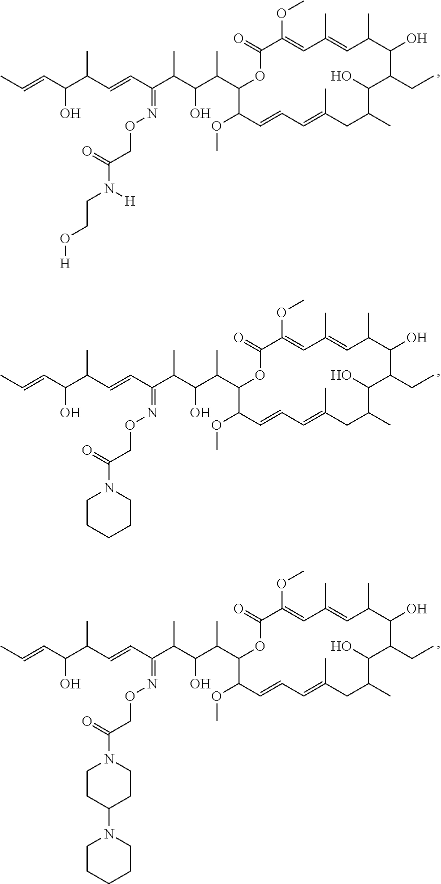 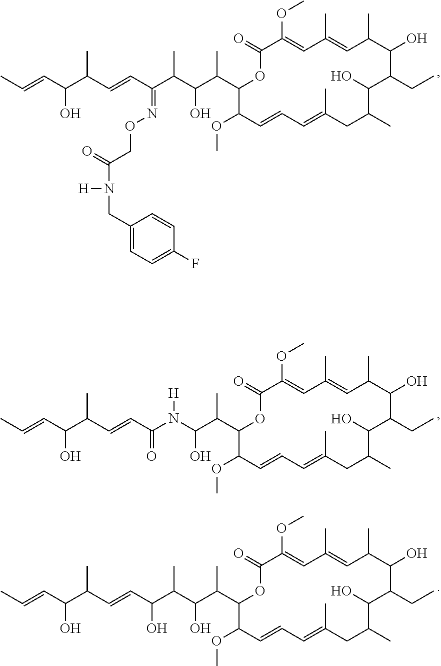
or a stereoisomeric form, tautomeric form, pharmaceutically acceptable salt or thereof

30. The method according to claim 27, wherein the compound of formula (1) is selected from: 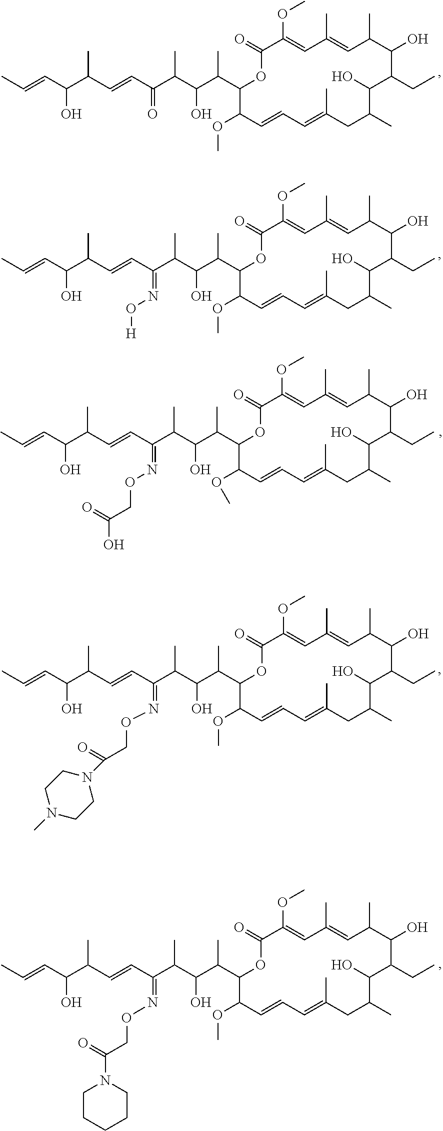 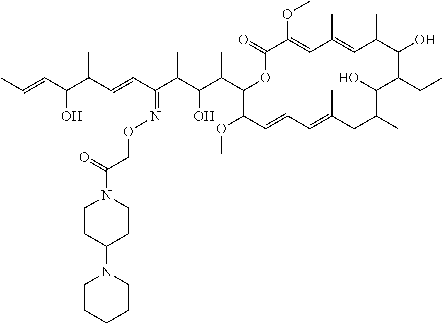
or a stereoisomeric form, tautomeric form, pharmaceutically acceptable salt thereof.

31. The method according to claim 27, wherein the inflammatory disorder is mediated by one or more inflammatory cytokines selected from Tumor Necrosis Factor-alpha (TNF-α), interferon-γ (IFN-γ) and interleukins (IL-1β, IL-2, IL-6 and IL-8).

32. The method according to claim 31, wherein the inflammatory disorder mediated by Tumor Necrosis Factor-alpha (TNF-α) is selected from the group consisting of inflammatory bowel disease, inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, septic shock, endotoxic shock, atherosclerosis, ischemia-reperfusion injury, coronary heart disease, vasculitis, amyloidosis, multiple sclerosis, sepsis, chronic recurrent uveitis, hepatitis C virus infection, malaria, ulcerative colitis, cachexia, psoriasis, plasmocytoma, endometriosis, Behcet's disease, Wegener's granulomatosis, meningitis, AIDS, HIV infection, autoimmune disease, immune deficiency, common variable immunodeficiency (CVID), chronic graft-versus-host disease, trauma and transplant rejection, adult respiratory distress syndrome, pulmonary fibrosis, recurrent ovarian cancer, lymphoproliferative disease, refractory multiple myeloma, myeloproliferative disorder, diabetes, juvenile diabetes, ankylosing spondylitis, skin delayed-type hypersensitivity disorders, Alzheimer's disease, systemic lupus erythematosus, and allergic asthma.

33. The method according to claim 31, wherein the inflammatory disorder mediated by interleukins (IL-1β, IL-2, IL-6 and IL-8) is selected from the group consisting of rheumatoid arthritis, osteoarthritis and autoimmune conditions.

34. The method according to claim 31, wherein the inflammatory disorder mediated by interferon-γ (IFN-γ) is selected from the group consisting of rheumatoid arthritis, osteoarthritis and autoimmune conditions.

35. The method according to claim 32, wherein the inflammatory disorder is selected from the group consisting of inflammatory bowel disease, inflammation, rheumatoid arthritis, juvenile rheumatoid arthritis, psoriatic arthritis, osteoarthritis, refractory rheumatoid arthritis, chronic non-rheumatoid arthritis, osteoporosis/bone resorption, Crohn's disease, ulcerative colitis, refractory multiple myeloma, myeloproliferative disorder, psoriasis, common variable immunodeficiency (CVID), skin delayed-type hypersensitivity disorders, systemic lupus erythematosus, allergic asthma and ankylosing spondylitis.

36. The method according to claim 35, wherein the inflammatory disorder is rheumatoid arthritis.

37. The method according to claim 35, wherein the inflammatory disorder is ulcerative colitis.

38. A method for monitoring drug response in a patient with an inflammatory disorder treated with a compound of formula (1) comprising determining the expression of one or more of genes selected from CEBPα, CEBPβ, CEBPδ, IL-1β, IL-6, GBP-1, MMP 13, MyD88, BCL2 and cMyc in a test sample obtained from the patient treated with said compound of formula (1) and comparing it to the expression of the same one or more of CEBPα, CEBPβ, CEBPδ, IL-1β, IL-6, GBP-1, MMP 13, MyD88, BCL2 and cMyc in a sample obtained from the patient before treatment with the compound of formula (1).

39. The method according to claim 38, wherein a change in the expression of the one or more of genes selected from CEBPα, CEBPβ, CEBPδ, IL-1β, IL-6, GBP-1, MMP 13, MyD88, BCL2 and cMyc after treatment with the compound of formula (1) is indicative of a drug response.

40. The method according to claim 38, wherein the expression of one or more of genes selected from CEBPα, CEBPβ, CEBPδ, IL-1β, IL-6, GBP-1, MMP 13, MyD88, BCL2 and cMyc is down-regulated after treatment with the compound of formula (1).

41. The method according to claim 38, wherein the compound of formula (1) is 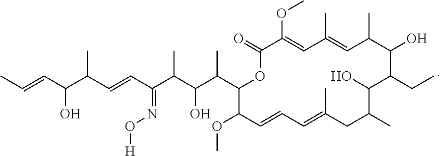
or a stereoisomeric form, or a tautomeric form, or a pharmaceutically acceptable salt, thereof.

42. The method according to claim 38, wherein the inflammatory disorder is mediated by CREB pathway.
